(19) United States
(12) Patent Application Publication
(10) Pub. No.: US 2015/0275238 A1
(43) Pub. Date: Oct. 1, 2015

(54) GENETICALLY ENGINEERED MICROBES AND METHODS FOR CONVERTING ORGANIC ACIDS TO ALCOHOLS

(71) Applicants: UNIVERSITY OF GEORGIA RESEARCH FOUNDATION, INC., Athens, GA (US); NORTH CAROLINA STATE UNIVERSITY, Raleigh, NC (US)

(72) Inventors: Robert M. Kelly, Raleigh, NC (US); Michael W.W. Adams, Athens, GA (US); Gerrit J. Schut, Athens, GA (US); Mirko Basen, Frankfurt (DE)

(21) Appl. No.: 14/670,601

(22) Filed: Mar. 27, 2015

Related U.S. Application Data
(60) Provisional application No. 61/971,609, filed on Mar. 28, 2014.

(51) Int. Cl. C12P 7/22; C12P 7/04; C12P 7/16; C12P 7/18; C12P 7/02; C12P 7/06

(52) U.S. Cl. CPC C12P 7/22; C12P 7/02; C12P 7/065; C12P 7/16; C12P 7/18; C12P 7/04

(57) ABSTRACT

Disclosed herein are genetically engineered microbes. In one embodiment, a genetically engineered microbe includes a metabolic pathway for the production of an alcohol from an organic acid. For instance, a genetically engineered microbe converts acetate, butyrate, propionate, isobutyrate, valerate, isovalerate, caproate, or phenylacetate, to Ethanol, Butanol, Propanol, Isobutanol, 1-Pentanol, Isoamylalcohol, 1-Hexanol, Phenylethanol, respectively. Also provided herein are methods of using the microbes.

### CROSS-REFERENCE TO RELATED APPLICATIONS

This application claims the benefit of U.S. Provisional Application Ser. No. 61/971,609, filed Mar. 28, 2014, which is incorporated by reference herein.

### GOVERNMENT FUNDING

The present invention was made with government support under DE-AR0000081 and DE-13502-06ER64304, awarded by the Department of Energy, and CBET-1264052/CBET-1264053, awarded by the National Science Foundation. The government has certain rights in the invention.

### BACKGROUND

Production of alcohol-based biofuels from renewable feedstocks is currently achieved by only a very limited number of metabolic pathways (Nielsen et al., 2013, Curr Opin Biotechnol 24(3):398-404; Peralta-Yahya et al., 2010, Biotechnol J 5(2):147-162). The US bioethanol industry depends on glucose conversion by yeast wherein pyruvate (C₃) is decarboxylated to acetaldehyde and then reduced to ethanol (C₂) by a monofunctional alcohol dehydrogenase. The other major pathway is found in some anaerobic bacteria, wherein glucose-derived pyruvate is oxidized to acetyl-CoA, and this is further reduced to ethanol by a bifunctional alcohol dehydrogenase (AdhE) (Carere et al., 2012, BMC Microbiol 12:295; Chang et al., 2011, Appl Microbiol Biotechnol, 92(1):13-27). Recently, there has been increasing interest in microorganisms that produce longer-chain alcohols (>C₂), which have superior characteristics as fuel molecules compared with ethanol, to replace fossil fuels (Nielsen et al., 2013, Curr Opin Biotechnol 24(3):398-404; Peralta-Yahya et al., 2010, Biotechnol J 5(2):147-162). In this case, glucose conversion requires microbial strains engineered to produce one specific alcohol at a time. For example, the acetone-butanol-ethanol fermentation pathway, found in some Clostridia, has been adapted in yeast, Escherichia coli, and a few other bacteria (Peralta-Yahya et al., 2010, Biotechnol J 5(2):147-162), Branduardi et al. 2014, Eng Life Sci, 14(1):16-26) to produce isopropanol or 1-butanol. Similarly, n-propanol, 2-methyl-1-butanol, 3-methyl-1-butanol, and 1-butanol are side products of amino acid fermentation by yeast (Peralta-Yahya et al., 2010, Biotechnol J 5(2):147-162), and modified pathways have been expressed in E. coli to produce a specific alcohol (Atsumi et al., 2008, Nature, 451(7174):86-89). In addition, isopentanol can be produced by a variation of the isoprenoid biosynthesis pathway in engineered E. coli Peralta-Yahya et al., 2010, Biotechnol J 5(2):147-162).

Production of bioalcohols at temperatures above 70° C. has several advantages over ambient-temperature processes, including lower risk of microbial contamination, higher diffusion rates, and lower cooling and distillation costs (Taylor et al., 2009, Trends Biotechnol, 27(7):398-405). However, very few microorganisms able to grow at such temperatures are able to generate ethanol from sugar (Wiegel et al., 1981, Arch Microbiol, 128(4):343-348; Yao et al., 2010, J Mol Microbiol Biotechnol, 19(3):123-133; Svetlitchnyi et al., 2013, Biotechnol Biofuels, 6(1):31), and no bacterium growing above 70° C. produces an alcohol other than ethanol. In addition, no member of the domain Archaea is known to produce any alcohol as a major product, regardless of growth temperature.

### SUMMARY

Decreasing fossil fuel reserves have accelerated efforts for bioalcohol production from renewable feedstocks using fermentative microorganisms as whole cell catalysts. Ethanol production is well established in yeast in which the end product of glycolysis, pyruvate, is decarboxylated to acetaldehyde and reduced to ethanol by an alcohol dehydrogenase (ADH). In contrast, some anaerobic bacteria oxidize pyruvate to acetyl-coenzyme A, which is subsequently reduced to ethanol by a bifunctional aldehyde/alcohol dehydrogenase (AdhE). The archaeon Pyrococcus furiosus ferments sugars to the end products of acetate, carbon dioxide, and hydrogen gas. The acetate is not used as a substrate for other reactions, and is excreted by the cell. The expression of AdhE in an engineered P. furiosus was expected to result in the production of ethanol through the action of AdhE on acetyl-coenzyme A, the normal precursor to acetate. Surprisingly, very little ethanol was produced. In contrast, the expression of AdhA (primary alcohol dehydrogenase) in P. furiosus was not expected to yield increased ethanol levels because the substrate of AdhA, acetaldehyde, is not normally produced in the necessary quantity by P. furiosus; however, AdhA expression in P. furiosus resulted in a dramatic and unexpected increase in ethanol production. Further analysis indicated that the enzyme aldehyde ferredoxin oxidoreductase (AOR), which had been proposed to convert to acetate the trace amounts of various aldehydes that are produced during normal peptide metabolism by P. furiosus, actually also worked in the reverse direction to reduce acetate to acetaldehyde. Thus, AOR and AdhA function together to route acetate to ethanol. Further, this pair of enzymes was also found to catalyze the conversion of other organic acids to their respective alcohols.

Presented herein is an alternative synthetic microbial pathway for alcohol production. In one exemplary embodiment, the hyperthermophilic archaeon Pyrococcus furiosus was genetically modified to express a primary alcohol dehydrogenase (AdhA) (from the thermophilic ethanol-producing bacterium Thermoanaerobacter strain X514). The mutant strain uses aliphatic, branched-chain and aromatic carboxylic acids as electron acceptors to produce the corresponding alcohol without the involvement of the activated CoA ester. The recombinant P. furiosus strain produces up to 40 mM alcohol (in non-optimized batch setting) from the corresponding acid at temperatures near 75° C. This takes advantage of the use of temperature as an inducer of heterologously-expressed enzymes in P. furiosus (Kelly et al., WO 2013/067326). This is also the first report of significant alcohol production by a hyperthermophilic microorganism or an archaeon. Furthermore, we show that the reduction of the carboxylic acids to alcohols is carried out by the native P. furiosus enzyme aldehyde-ferredoxin oxidoreductase (AOR) that is linked to the heterologously-expressed alcohol dehydrogenase. The synthetic AOR-AdhA pathway takes advantage of the low redox potential of P. furiosus metabolism to drive the AOR reaction and the high affinity alcohol dehydrogenase from T X514 to pull the reaction toward alcohol formation. This synthetic pathway provides a new perspective on microbial redox metabolism, and provides a method for alcohol production from carboxylic acids in different fermentation settings. The use of a synthetic pathway using AOR and AdhA enzymes, also from different gene donors, is expected to be extendable to other anaerobic microorganisms as hosts.

Disclosed herein are genetically engineered microbes. In one embodiment, a genetically engineered microbe includes a metabolic pathway for the production of an alcohol from an organic acid under suitable conditions. The metabolic pathway includes an enzyme having aldehyde ferredoxin oxidoreductase (AOR) activity and an enzyme having alcohol dehydrogenase (AdhA) activity. An example of suitable conditions includes a temperature of at least 50° C., at least 60° C., or at least 70° C. In one embodiment, the conditions include an anaerobic environment. In one embodiment, the organic acid is acetate, butyrate, propionate, isobutyrate, valerate, isovalerate, caproate, phenylacetate, benzoic acid, Lactate, or 3-Hydroxypropionate.

The enzyme having AOR activity may be endogenous or exogenous, and the enzyme having AdhA activity may be endogenous or exogenous. In those embodiments where the AOR activity is exogenous a coding region encoding the activity may be integrated into the chromosome. In those embodiments where the AdhA activity is exogenous a coding region encoding the activity may be integrated into the chromosome. In one embodiment, the metabolic pathway includes reduction of the organic acid coupled to oxidation of ferredoxin.

The genetically engineered microbe may be an archaeon or a bacterium. In one embodiment, the genetically engineered microbe is Pyrococcus furiosus, Thermococcus spp. Caldicellulosiruptor spp., Thermoanaerobacter spp., Thermoanaerobacterium spp., Moorella spp., or Clostridium spp.

In one embodiment, the genetically engineered microbe also includes a carbon monoxide dehydrogenase (CODH) activity. The CODH activity may be a complex of polypeptides. In one embodiment, the complex includes 16 polypeptides, and the 16 polypeptides includes amino acid sequences filed in Genbank under accession numbers 212009123, 212009124, 212009125, 212009126, 212009127, 212009128, 212009129, nucleotides 948282 to 948778 of GI number 212008101, 212009130, 212009131, 212009132, 212009133, 212009134, 212009135, 212009136, and 212009137. In one embodiment, one or more of the 16 polypeptides of the complex are structurally similar to the amino acid sequences of the polypeptides disclosed at these Genbank accession numbers.

Also disclosed herein are methods. In one embodiment, the method is for producing an alcohol. The method includes culturing a genetically engineered microbe disclosed herein under conditions suitable to produce an alcohol. In one embodiment, the culturing includes an incubation temperature of at least 70° C. In one embodiment, the culturing includes a carbon source selected from a hexose carbohydrate, such as glucose, a pentose carbohydrate, or a combination thereof. In one embodiment, the culturing includes a plant biomass, such as a lignocellulosic biomass. In one embodiment, the alcohol produced is Ethanol, Butanol, Propanol, Isobutanol, 1-Pentanol, Isoamylalcohol, 1-Hexanol, Phenylethanol, Benzyl alcohol, 1,2-Propanediol, or 1,3-Propanediol. In one embodiment, the method includes isolating the alcohol.

As used herein, a “microbe” is a single celled organism that is a member of the domain Archaea or a member of the domain Bacteria. As used herein, a “genetically engineered microbe” and “genetically modified microbe” refers to a microbe that has been altered “by the hand of man,” for instance, by the introduction of an exogenous polynucleotide. For example, a microbe is a genetically engineered microbe by virtue of introduction into a suitable microbe of an exogenous polynucleotide. “Genetically engineered microbe,” also refers to a microbe that has been genetically manipulated such that endogenous nucleotides have been altered. For example, a microbe is a genetically engineered microbe by virtue of introduction into a suitable microbe of an alteration of endogenous nucleotides. For instance, an endogenous coding region could be deleted or mutagenized. Such mutations may result in a polypeptide having a different amino acid sequence than was encoded by the endogenous polynucleotide. Another example of a genetically engineered microbe is one having an altered regulatory sequence, such as a promoter, to result in altered expression of an operably linked endogenous coding region.

As used herein, an “extreme thermophile” is a member of the domain Bacteria or Archaea that grows optimally in environments of at least 70° C. As used herein, a “hyperthermophile” is a member of the domain Bacteria or Archaea that grows optimally in environments of at least 80° C.

As used herein, the term “polynucleotide” refers to a polymeric form of nucleotides of any length, either ribonucleotides or deoxynucleotides, and includes both double- and single-stranded DNA and RNA. A polynucleotide may include nucleotide sequences having different functions, including for instance coding sequences, and non-coding sequences such as regulatory sequences. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment.

An “exogenous polynucleotide” refers to a foreign polynucleotide that is not normally present in a microbe. An exogenous polynucleotide may be separate from the genomic DNA of a cell (e.g., it may be a vector, such as a plasmid), or an exogenous polynucleotide may be integrated into the genomic DNA of a cell. A regulatory region, such as a promoter, that is present in the genomic DNA of a microbe but has been modified to have a nucleotide sequence that is different from the promoter normally present in the microbe is also considered an exogenous polynucleotide. An exogenous polynucleotide may encode an exogenous polypeptide or an endogenous polypeptide. As used herein, the term “endogenous polynucleotide” refers to a polynucleotide that is normally or naturally found in a microbe. An “endogenous polypeptide” is also referred to as a “native polypeptide,” and an “endogenous polynucleotide” is also referred to as a “native polynucleotide.” An “endogenous polynucleotide” is also referred to as a “native polynucleotide.”

As used herein, the term “polypeptide” refers broadly to a polymer of two or more amino acids joined together by peptide bonds. The term “polypeptide” also includes molecules which contain more than one polypeptide joined by disulfide bonds, ionic bonds, or hydrophobic interactions, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring.

An “exogenous polypeptide” refers to a foreign polypeptide, i.e., a polypeptide that is not normally present in a microbe. An “endogenous polypeptide” refers to a polypeptide that is normally present in a microbe. Since an exogenous polynucleotide may include, in some embodiments, a polynucleotide that is normally present in a microbe but is operably linked to a regulatory region to which it is not normally operably linked, in some embodiments an exogenous polynucleotide may encode an endogenous polypeptide. As used herein, the term “endogenous polynucleotide” refers to a polypeptide that is normally or naturally found in a microbe. An “endogenous polypeptide” is also referred to as a “native polypeptide.”

As used herein, “heterologous amino acid sequence” refers to amino acid sequences that are not normally present as part of a polypeptide present in a wild-type cell. For instance, “heterologous amino acid sequence” includes extra amino acids at the amino terminal end or carboxy terminal of a polypeptide that are not normally part of a polypeptide.

As used herein, an “isolated” substance is one that has been removed from its natural environment, produced using recombinant techniques, or chemically or enzymatically synthesized. For instance, a polypeptide or a polynucleotide described herein can be isolated. With respect to a product produced using a method described herein, such as an alcohol, “isolated” refers to removal of the product from the medium in which it was produced by a genetically engineered microbe. Preferably, a substance is purified, i.e., is at least 60% free, preferably at least 75% free, and most preferably at least 90% free from other components with which it is naturally associated, or from other components present in the medium in which it was produced.

As used herein, the terms “coding region,” “coding sequence,” and “open reading frame” are used interchangeably and refer to a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Non-limiting examples of regulatory sequences include promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

The terms “complement” and “complementary” as used herein, refer to the ability of two single stranded polynucleotides to base pair with each other, where an adenine on one strand of a polynucleotide will base pair to a thymine or uracil on a strand of a second polynucleotide and a cytosine on one strand of a polynucleotide will base pair to a guanine on a strand of a second polynucleotide. Two polynucleotides are complementary to each other when a nucleotide sequence in one polynucleotide can base pair with a nucleotide sequence in a second polynucleotide. For instance, 5′-ATGC and 5′-GCAT are complementary. The term “substantial complement” and cognates thereof as used herein, refer to a polynucleotide that is capable of selectively hybridizing to a specified polynucleotide under stringent hybridization conditions. Stringent hybridization can take place under a number of pH, salt and temperature conditions. The pH can vary from 6 to 9, preferably 6.8 to 8.5. The salt concentration can vary from 0.15 M sodium to 0.9 M sodium, and other cations can be used as long as the ionic strength is equivalent to that specified for sodium. The temperature of the hybridization reaction can vary from 30° C. to 80° C., preferably from 45° C. to 70° C. Additionally, other compounds can be added to a hybridization reaction to promote specific hybridization at lower temperatures, such as at or approaching room temperature. Among the compounds contemplated for lowering the temperature requirements is formamide. Thus, a polynucleotide is typically substantially complementary to a second polynucleotide if hybridization occurs between the polynucleotide and the second polynucleotide. As used herein, “specific hybridization” refers to hybridization between two polynucleotides under stringent hybridization conditions.

Conditions that “allow” an event to occur or conditions that are “suitable” for an event to occur, such as an enzymatic reaction, or “suitable” conditions are conditions that do not prevent such events from occurring. Thus, these conditions permit, enhance, facilitate, and/or are conducive to the event. Such conditions, known in the art and described herein, may depend upon, for example, the enzyme being used.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Sugar fermentation coupled to alcohol production by P. furiosus strain A. Glucose from sugars is oxidized to acetate and CO₂ and ferredoxin is reduced by GAPOR and POR. In the engineered strain A, acetate is reduced to acetaldehyde by AOR and then reduced to ethanol by the heterologously expressed AdhA from Thermoanaerobacter strain X514. The redox balance is maintained by the production of H₂ by the energy-conserving, membrane-bound hydrogenase (MBH) and H₂ oxidation by SHI. Organic acids added exogenously are reduced to the corresponding aldehyde and alcohol by AOR and AdhA, respectively, using reductant generated by glucose oxidation.

FIG. 2. Formation of ethanol from sugars by engineered P. furiosus strains. (A) Genetic constructs with Thermoanaerobacter strain X514 adhE and/or adhA for genome insertion into P. furiosus strain COM1. (B) Specific activities of AdhE (open bars) and AdhA (solid bars) in cell-free extracts of P. furiosus strains EA, E, and A, and parent strain COM1 grown at 72° C. (C) Ethanol (left bar of each pair) and acetate (right bar of each pair) produced after 4 d incubation at 72° C. with maltose (5 g·L⁻¹) as the carbon source. (D) Time course of ethanol and acetate production in strain A (▴) and COM1 () at 72° C. with cellobiose (5 g·L⁻¹) as the carbon source. After 4 d, ˜35% of the cellobiose was converted to ethanol. Experimental data represent the average of three independently prepared cell extracts or cultures (n=3; ±SD).

FIG. 3. Maps of plasmids used in this study. (A) pMB403SLP, (B) pMB404SLP and (C) pMB407SLP were used to transform Pyrococcus furiosus strain COM1, using pyrF as selective marker. They contained the gene(s) adhE and/or adhA from Thermoanaerobacter strain X514 under the control of the strong constitutive promoter Pslp. They were inserted between the converging genes PF0574 and PF0575 (Keller et al., 2013, Proc Natl Acad Sci USA 110:5840-5845). See Materials and Methods of Example 1 for details.

FIG. 4. Formation of 13C-ethanol from 13C-acetate by P. furiosus strain A. Percentage ratio of ¹³C vs. total C (¹²C+¹³C) determined in ethanol (blue triangles) and acetate (red circles) in cultures incubated at 72° C. on unlabeled maltose (5 g L-1, 15 mM) supplied with 8 mM of double-labeled ¹³C-acetate. Average of three independent cultures (n=3; ±SD).

FIG. 5. Effect of aor deletion on ethanol formation from maltose. Formation of ethanol (left bar of each pair) and acetate (right bar of each pair) in Strains COM1, A (harboring adhA) and AΔaor (strain A with aor gene deleted) when incubated with maltose (5 g L-1, 15 mM) at 72° C. for 3 days (n=3; ±SD).

FIG. 6. Reduction of organic acids to alcohols by P. furiosus strain A. (A) Various organic acids were reduced to the corresponding alcohols (black bars) with concomitant production of acetate (middle bar of each set) and ethanol (right bar of each set) during incubation of strain A with maltose for 5 d at 72° C. (B) Equimolar formation of butanol (squares) from butyrate and acetate (circles) from maltose by a 10-fold concentrated cell suspension, with only minor amounts of ethanol (triangles) formed. (C) Effect of aor deletion on butanol formation in strain A. (D) Effect of hydrogen on the oxidation of pyruvate to acetate and reduction of butyrate to butanol by a 10-fold concentrated cell suspension of strain A. All experimental data represent the average of three independent cultures (n=3; ±SD).

FIG. 7. Conversion of sugars to ethanol and butyrate to butanol by P. furiosus. (A) Product formation from maltose in strain COM1c. (B) Formation of ethanol from maltose by strain A. (C) Formation of butanol from butyrate by strain A. Concentration of metabolites are represented as follows: dark circles, acetate; triangles, ethanol; lighter circles, H2 (represented as mmol per L medium); open black circles, total cell protein (μg cell protein per mL medium); diamonds, butyrate; squares, butanol.

FIG. 8. The T. onnurineus Codh complex and vector for insertion of the Codh expression construct into the P. furiosus chromosome. (A) A schematic representation of the Ton CODH complex encoded by TON_{—}1017-1031. This shows subunits of carbon monoxide dehydrogenase (Codh), membrane-bound hydrogenase (Mbh), and the Na+/H+ transporter (Mrp). CooC (TON_{—}1019) encodes the maturation protein for CooS, and the function of CooX encoded by TON_{—}1020 is not known. (B) The BAC-based vector pGL058 containing the Codh operon TON_{—}1017-1031 gene cluster (Codh, TON_{—}1017-20; Mbh, TON_{—}1021-25, green; Mrp, TON_{—}1025-31) under transcriptional control of the promoter for P. furiosus membrane-bound hydrogenase Pmbh1; the pyrF marker cassette and 5′ and 3′ homologous recombination regions used for targeted insertion into the P. furiosus chromosome. The BAC vector backbone features are shown in grey (from left to right: cat marker, oris, repE, sopA, sopB, sopC and cos).

FIG. 9. CO as source of reductant for conversion of organic acids to alcohols by P. furiosus strain A/Codh. (A) Isobutanol formation (squares) from isobutyrate (diamonds) in the presence of CO (diamonds) by cultures of strain A/Codh grown with maltose as the carbon source at 72° C. (n=2; ±SD). (B) CO oxidation linked to organic acid reduction by P. furiosus strain A/Codh. The CODH complex oxidizes CO with the production of H₂ and also reduces ferredoxin to provide low potential electrons to the AOR reaction. NADPH for the AdhA reaction is supplied by H₂ oxidation by SHI.

FIG. 10. Reduction of organic acids to alcohols by Pyrococcus furiosus strain A/Codh in the absence of CO. Strain A/Codh was incubated at 75° C. under argon and the production and utilization of alcohols and organic acids were determined. Less than 1 mM ethanol was produced under these conditions.

FIG. 11. Reduction of organic acids to alcohols by Pyrococcus furiosus strain A/Codh cell suspensions. Formation of hydrogen (circles) and of isobutanol (squares) from isobutyrate in the presence (filled symbols) or absence (open symbols, dotted lines) of CO as the only electron donor by a 10-fold concentrated cell suspension (˜3×10⁹ cells/ml).

FIG. 12. Substrate specificity and temperature optimum of T. X514 AdhA. (A) Relative specific activity of AdhA in the cell extracts of strain A with various aldehydes. (B) Specific activity of AdhA at different temperatures (n=3; ±SD).

FIG. 13. Michaelis-Menten kinetics of T. X514 AdhA. Specific activity of AdhA in the cell extracts of P. furiosus strain A at different substrate concentrations of (A) butyraldehyde, (B) acetaldehyde and (C) NADPH.

FIG. 14. Temperature dependence of the AOR/AdhA synthetic pathway. (A) Activity of AdhA in cell extracts of strain A grown at different temperatures. (B) Conversion of butyrate (40 mM; diamonds) to butanol (squares) and acetate (circles) by strain A incubated at different temperatures in the presence of maltose (15 mM). Only minor amounts of ethanol (triangles) were formed. All experimental data represent the average of three independent cultures (n=3; ±SD).

FIG. 15. An example of an aldehyde ferredoxin oxidoreductase (AOR) polypeptide (SEQ ID NO:1), and an example of an alcohol dehydrogenase (AdhA) polypeptide (SEQ ID NO:2).

FIG. 16. Multiple protein alignment of SEQ ID NO:1 (AOR), with Halanaerobium hydrogeniformans (AOR, SEQ ID NO:3), Geobacter sulfurreducens (AOR, SEQ ID NO:4), and Clostridium ljungdahlii (AOR, SEQ ID NO:6). An * (asterisk) indicates positions which have a single, fully conserved residue; a : (colon) indicates conservation between groups of strongly similar properties—scoring>0.5 in the Gonnet PAM 250 matrix; a . (period) indicates conservation between groups of weakly similar properties—scoring=<0.5 in the Gonnet PAM 250 matrix.

FIG. 17. Multiple protein alignment of SEQ ID NO:2 (AdhA), with Clostridium botulinum (Adh, SEQ ID NO:9), and Desulfotomaculum ruminis (Adh, SEQ ID NO:10). An * (asterisk) indicates positions which have a single, fully conserved residue; a : (colon) indicates conservation between groups of strongly similar properties—scoring>0.5 in the Gonnet PAM 250 matrix; a . (period) indicates conservation between groups of weakly similar properties—scoring=<0.5 in the Gonnet PAM 250 matrix.

### DETAILED DESCRIPTION OF ILLUSTRATIVE EMBODIMENTS

Provided herein are genetically engineered microbes that include a synthetic metabolic pathway that converts an organic acid to an alcohol. Examples of organic acids include carboxylic acids. In one embodiment, a carboxylic acid includes a carboxyl group, —COOH, and a carbon-containing organic compound. In one embodiment, the organic compound is an alkyl group, an aryl group, an alkenyl group, or an alkynyl group. The term “alkyl group” means a saturated linear or branched monovalent hydrocarbon group including, for example, methyl, ethyl, n-propyl, isopropyl, tert-butyl, amyl, heptyl, and the like. The term “aryl group” means a mono- or polynuclear aromatic hydrocarbon group. The term “alkenyl group” means an unsaturated, linear or branched monovalent hydrocarbon group with one or more olefinically unsaturated groups (i.e., carbon-carbon double bonds), such as a vinyl group. The term “alkynyl group” means an unsaturated, linear or branched monovalent hydrocarbon group with one or more carbon-carbon triple bonds. A linear or branched hydrocarbon group may include at least 1, at least 2, at least 3, at least 4, at least 5, at least 6, at least 7, at least 8, at least 9, at least 10, at least 11, or at least 12 carbon atoms. Non-limiting examples of organic acids, and the alcohol produced using the organic acid, are shown in Table 1.

TABLE 1

| Organic acid | Alcohol produced |
|---|---|
| acetate | Ethanol |
| butyrate | Butanol |
| propionate | Propanol |
| isobutyrate | Isobutanol |
| valerate | 1-Pentanol |
| isovalerate | Isoamylalcohol |
| caproate | 1-Hexanol |
| phenylacetate | Phenylethanol |
| benzoic acid | Benzyl alcohol |
| Lactate | 1,2-Propanediol |
| 3-Hydroxypropionate | 1,3-Propanediol |

The metabolic pathway described herein for the production of an alcohol from an organic acid includes two enzymes, an aldehyde ferredoxin oxidoreductase (AOR) that converts an organic acid to its corresponding aldehyde, and an alcohol dehydrogenase (AdhA) that converts the aldehyde to its corresponding alcohol. The genetically engineered microbe is modified to include the metabolic pathway by addition of a polynucleotide encoding an AOR, addition of a polynucleotide encoding an AdhA, or addition of polynucleotides encoding an AOR and an AdhA. Thus, in one embodiment, the genetically engineered microbe includes an endogenous AOR and an exogenous AdhA. In one embodiment, the genetically engineered microbe includes an exogenous AOR and an endogenous AdhA. In one embodiment, the genetically engineered microbe includes an exogenous AOR and an exogenous AdhA.

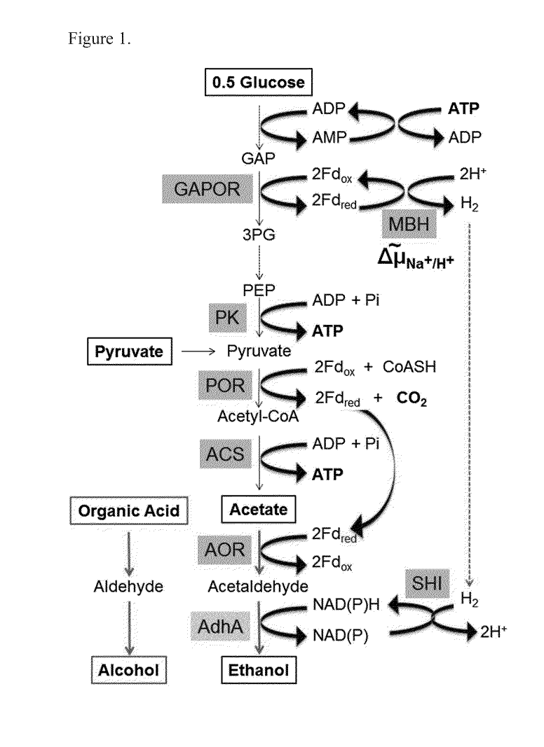

As used herein, “aldehyde ferredoxin oxidoreductase” or “AOR” refers to a polypeptide that, regardless of its common name or native function, catalyses the conversion of an organic acid listed in Table 1 to the corresponding aldehyde, for instance, the conversion of acetate to acetaldehyde (see FIG. 1). In one embodiment, an AOR polypeptide that catalyses the conversion of an organic acid to the corresponding aldehyde couples the conversion to the oxidation of ferredoxin. A polypeptide catalysing such a conversion has AOR activity, and a polypeptide having AOR activity is referred to herein as an AOR polypeptide. An AOR polypeptide is enzymatically active at a temperature of at least 60° C., at least 70° C., at least 80° C., or at least 90° C. In one embodiment, such an enzyme is a member of the group having Enzyme Commission (EC) number EC 1.2.7.5. Enzymes having AOR activity are readily available from, for instance, PF0346 from Pyrococcus furiosus (Roy et al., 1999, J. Bacteriol., 181(4):1171), Moorella thermoacetica (White et al. 1989, Eur. J. Biochem., 184: 89-96), Thermoanaerobacter pseudethanolicus, and other members of the order Thermococcales. In one embodiment, the polypeptide having AOR activity is, or is structurally similar to, a reference polypeptide that includes the amino acid sequence of SEQ ID NO:1.

As used herein, “alcohol dehydrogenase” or “AdhA” refers to a polypeptide that, regardless of its common name or native function, catalyses the conversion of an aldehyde to its corresponding alcohol, for instance, the conversion of acetaldehyde to ethanol (see FIG. 1). A polypeptide catalysing such a conversion has AdhA activity, and a polypeptide having AdhA activity is referred to herein as an AdhA polypeptide. An AdhA polypeptide is enzymatically active at a temperature of at least 60° C., at least 70° C., at least 80° C., or at least 90° C. In one embodiment, such an enzyme is a member of the group having Enzyme Commission (EC) number EC:1.1.1.2, or EC1.1.1.1, or 1.1.1.71. Enzymes having AdhA activity are readily available from, for instance, Thermoanaerobacter strain X514 (Roh, et al., 2002, Appl. Environ. Microbiol. 68:6013-6020), Thermoanaerobacter mathranii (Yao and Mikkelsen, 2010, J. Mol. Microbiol. Biotechnol. 19:123-133), Thermoanaerobacter ethanolicus (Pei et al., 2010, Metab. Eng. 12: 420-428) and other members of the genus Thermanaerobacter. In one embodiment, the polypeptide having AdhA activity is, or is structurally similar to, a reference polypeptide that includes the amino acid sequence of SEQ ID NO:2.

As used herein, a polypeptide is “structurally similar” to a reference polypeptide if the amino acid sequence of the polypeptide possesses a specified amount of similarity and/or identity compared to the reference polypeptide. Structural similarity of two polypeptides can be determined by aligning the residues of the two polypeptides (for example, a candidate polypeptide and the polypeptide of, for example, SEQ ID NO:1 or SEQ ID NO:2) to optimize the number of identical amino acids along the lengths of their sequences; gaps in either or both sequences are permitted in making the alignment in order to optimize the number of identical amino acids, although the amino acids in each sequence must nonetheless remain in their proper order.

A pair-wise comparison analysis of amino acid sequences can be carried out using the BESTFIT algorithm in the GCG package (version 10.2, Madison, Wis.). Alternatively, polypeptides may be compared using the Blastp program of the BLAST 2 search algorithm, as described by Tatiana et al., 1999, FEMS Microbiol Lett, 174:247-250), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a polypeptide described herein may be selected from other members of the class to which the amino acid belongs. For example, it is well-known in the art of protein biochemistry that an amino acid belonging to a grouping of amino acids having a particular size or characteristic (such as charge, hydrophobicity, and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2. Likewise, biologically active analogs of a polypeptide containing deletions or additions of one or more contiguous or noncontiguous amino acids that do not eliminate a functional activity of the polypeptide are also contemplated.

The crystal structure of at least one AOR is known (Chan et al., 1995, Science, 267: 1463-1469). The skilled person will recognize that the structure of the AOR can be used to help predict which amino acids may be substituted, and which sorts of substitutions (e.g., conservative or non-conservative) can be made to an AOR without altering the activity of the polypeptide. The skilled person will recognize that the AOR depicted at SEQ ID NO:1 can be compared to AOR polypeptides from other extreme thermophiles, such as hyperthermophiles, including YP_{—}183479.1 (Thermococcus kodakarensis KOD1), YP_{—}001664842.1 (Thermoanaerobacter pseudethanolicus ATCC 33223), WP010477063.1 (Thermococcus zilligii) or EJ3 YP002960279.1 (Thermococcus gammatolerans) using readily available algorithms such as Clustl to identify conserved regions of AOR polypeptides. The skilled person will also recognize that the AOR depicted at SEQ ID NO:1 can be compared to AOR polypeptides from mesophiles using readily available algorithms such as Clustl to identify regions of AOR polypeptides that are conserved between mesophiles and thermophilic microbes. Examples of AORs from mesophiles include Halanaerobium hydrogeniformans (Genbank accession YP_{—}003994531), Clostridium ljungdahlii DSM 13528 (Genbank accession YP_{—}003780185), and Geobacter sulfurreducens PCA (Genbank accession NP_{—}951964). Using this information the skilled person will be able to readily predict with a reasonable expectation that certain conservative substitutions to an AOR such as SEQ ID NO:1 will not decrease activity of the polypeptide.

The skilled person will recognize that the AdhA depicted at SEQ ID NO:2 can be compared to AdhA polypeptides from other extreme thermophiles, such as hyperthermophiles, including Thermoanaerobacter mathranii) (Genbank accession YP_{—}003677778.1), Thermococcus onnurineus), (Genbank accession YP_{—}002306928.1), Thermoanaerobacter wiegelii Rt8.B1), (Genbank accession YP_{—}004819456.1), Thermoanaerobacter siderophilus (Genbank accession WP_{—}006570081.1), Thermoanaerobacter mathranii subsp. mathranii str. A3 (Genbank Accession YP_{—}003677778.1) using readily available algorithms such as Clustl to identify conserved regions of AdhA polypeptides. The skilled person will also recognize that the AdhA depicted at SEQ ID NO:2 can be compared to AdhA polypeptides from mesophiles using readily available algorithms such as Clustl to identify regions of AdhA polypeptides that are conserved between mesophiles and thermophilic microbes. Examples of AdhAs from mesophiles include Clostridium botulinum A str. ATCC 3502 (Genbank accession YP_{—}001253953) and Desulfotomaculum ruminis DSM 2154 (Genbank accession YP_{—}004544043). Using this information the skilled person will be able to readily predict with a reasonable expectation that certain conservative substitutions to an AdhA such as SEQ ID NO:2 will not decrease activity of the polypeptide.

In one embodiment, a polypeptide having an activity described herein can include a polypeptide with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to a reference amino acid sequence.

In one embodiment, a polypeptide having an activity described herein can include a polypeptide with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a reference amino acid sequence.

A candidate polypeptide (e.g., a polypeptide having structural similarity to a polypeptide described herein) may be isolated from a microbe, such as an extreme thermophile or a hyperthermophile. A candidate polypeptide may be produced using recombinant techniques, or chemically or enzymatically synthesized.

Guidance concerning how to make phenotypically silent amino acid substitutions is provided in Bowie et al. (1990, Science, 247:1306-1310), wherein the authors indicate proteins are surprisingly tolerant of amino acid substitutions. For example, Bowie et al. disclose that there are two main approaches for studying the tolerance of a polypeptide sequence to change. The first method relies on the process of evolution, in which mutations are either accepted or rejected by natural selection. The second approach uses genetic engineering to introduce amino acid changes at specific positions of a cloned gene and selects or screens to identify sequences that maintain functionality. As stated by the authors, these studies have revealed that proteins are surprisingly tolerant of amino acid substitutions. The authors further indicate which changes are likely to be permissive at a certain position of the protein. For example, most buried amino acid residues require non-polar side chains, whereas few features of surface side chains are generally conserved. Other such phenotypically silent substitutions are described in Bowie et al, and the references cited therein.

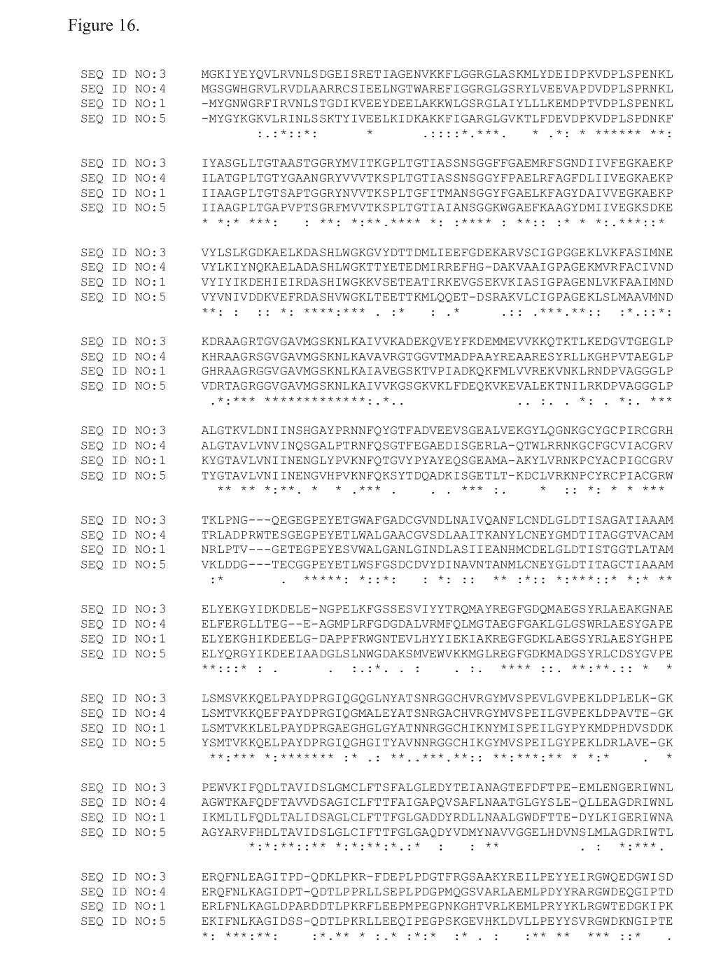

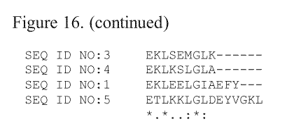

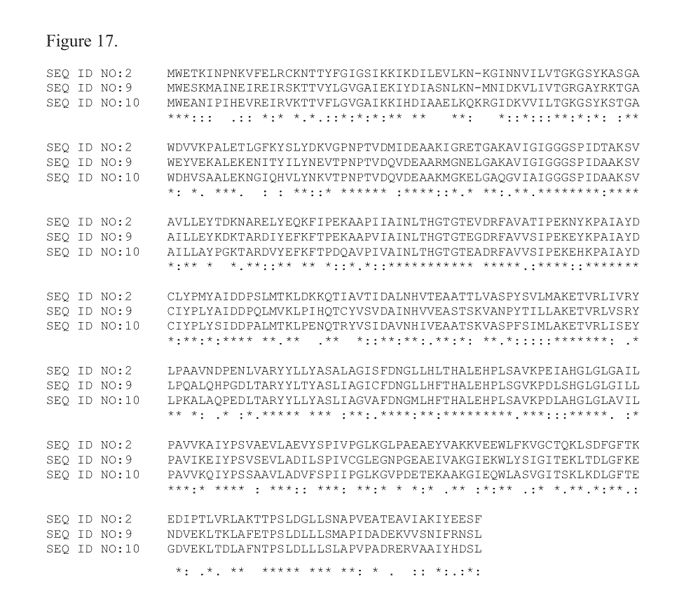

Guidance on how to modify the amino acid sequences of polypeptides disclosed herein is also provided at FIGS. 16 and 17. These figures show the amino acid sequences of polypeptides disclosed herein (SEQ ID NOs:1 and 2) in multiple protein alignments with other related polypeptides. Identical amino acids are marked with an asterisk (“*”), strongly conserved amino acids are marked with a colon (“:”), and weakly conserved amino acids are marked with a period (“.”). By reference to these figures, the skilled person can predict which alterations to an amino acid sequence are likely to modify enzymatic activity, as well as which alterations are unlikely to modify enzymatic activity.

A polypeptide described herein may be expressed as a fusion polypeptide that includes a polypeptide described herein and a heterologous amino acid sequence. The heterologous amino acid sequence may be present at the amino terminal end or the carboxy terminal end of a polypeptide, or it may be present within the amino acid sequence of the polypeptide. The number of heterologous amino acids may be, for instance, at least 5, at least 10, at least 15, at least 20, at least 25, at least 30, at least 35, or at least 40.

Also provided are isolated polynucleotides encoding the polypeptides described herein. For instance, a polynucleotide may have a nucleotide sequence encoding a polypeptide having the amino acid sequence shown in SEQ ID NOs:1 or 2, and an example of the class of nucleotide sequences encoding each polypeptide is disclosed herein as a coding region of Genbank accession NC_{—}003413.1 (the complement of nucleotides 358419 to 360236, open reading frame PF0346) and Genbank accession NC_{—}010320.1 (the complement of nucleotides 652326 to 654944, open reading frame Teth514_{—}0627), respectively. It should be understood that a polynucleotide encoding a polypeptide represented by one of the sequences disclosed herein, e.g., SEQ ID NOs:1 and 2, is not limited to the nucleotide sequence disclosed as a coding region of Genbank accession NC_{—}003413.1 (the complement of nucleotides 358419 to 360236) and Genbank accession NC_{—}010320.1 (the complement of nucleotides 652326 to 654944), but also includes the class of polynucleotides encoding such polypeptides as a result of the degeneracy of the genetic code. The class of nucleotide sequences encoding a selected polypeptide sequence is large but finite, and the nucleotide sequence of each member of the class may be readily determined by one skilled in the art by reference to the standard genetic code, wherein different nucleotide triplets (codons) are known to encode the same amino acid.

The metabolic pathway described herein for the production of an alcohol from an organic acid may include, in addition to an AOR polypeptide and an AdhA polypeptide, other enzymes for the production of an acid that can be used as a substrate for an AOR polypeptide. The enzymes may use peptides and/or amino acids to make an acid, which can be used as a substrate for an AOR polypeptide. In another embodiment, other enzymes in a microbe may break down sugars, such as glucose, into acetate, which is then used as a substrate for an AOR polypeptide. These enzymes are present in most microbes, and typically include the production of pyruvate, the end product of glycolysis. In one embodiment, the pathway includes one or more enzymes that couple the production of pyruvate to the reduction of ferredoxin. An example of such an enzyme includes, for instance, glyceraldehyde-3-phosphate (GAP) Fd oxidoreductase (GAPOR). While a microbe may make acids that are used by AOR and AdhA enzymes, a microbe may take up acids present in the medium and produce the corresponding alcohols via AOR and AdhA.

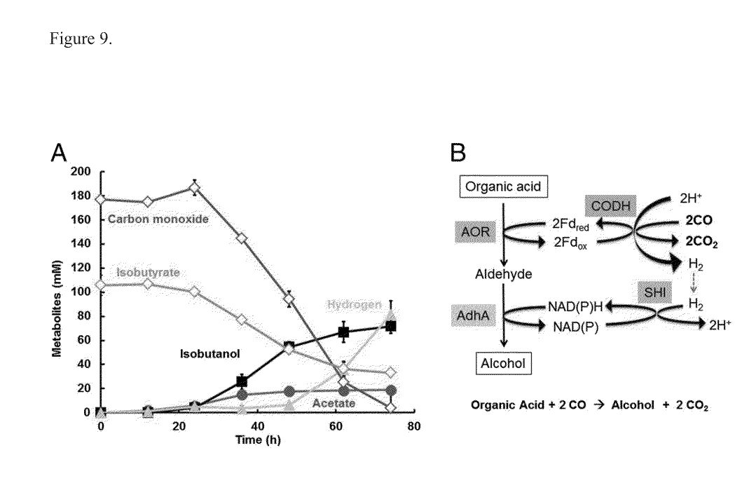

A genetically engineered microbe may also include an additional carbon monoxide dehydrogenase (CODH) activity. As used herein, “carbon monoxide dehydrogenase” or “CODH” refers to a polypeptide or complex of polypeptides that, regardless of its common name or native function, catalyses the oxidation of CO with the production of H₂ and CO₂ and also the reduction of ferredoxin (see FIG. 9B). A polypeptide or complex of polypeptides catalysing such a conversion has CODH activity, and a polypeptide or complex of polypeptides having CODH activity is referred to herein as a CODH polypeptide. A CODH polypeptide is enzymatically active at a temperature of at least 60° C., at least 70° C., at least 80° C., or at least 90° C. In one embodiment, the coding regions encoding the polypeptides having CODH activity is obtainable from anaerobic microbes such as members of the family Thermococcaceae, such as members of the genus Thermococcus including T. onnurineus. In one embodiment, a useful CODH complex of polypeptides can be obtained from T. onnurineus. The polypeptides are encoded by an operon of 16 coding regions, and is available at nucleotides 940254 to 953459 of Genbank accession number CP000855, GI number 212008101. Specifically, nucleotides 940254 to 940892 (open reading frame TON_{—}1017) encodes a 4Fe-4S ferredoxin, iron-sulfur binding domain protein (Genbank accession number ACJ16505.1, GI number 212009123). Nucleotides 940903 to 942795 (open reading frame TON_{—}1018) encodes a carbon-monoxide dehydrogenase, catalytic subunit (Genbank accession number ACJ16506.1, GI number 212009124).

Nucleotides 942863 to 943672 (open reading frame TON_{—}1019) encodes a hypothetical ATP-binding protein (Genbank accession number ACJ16507.1, GI number 212009125). Nucleotides 943673 to 943867 (open reading frame TON_{—}1020) encodes a hypothetical RNA-binding protein (Genbank accession number ACJ16508.1, GI number 212009126). Nucleotides 943872 to 945776 (open reading frame TON_{—}1021) encodes a hydrogenase 4, subunit 3 (Genbank accession number ACJ16509.1, GI number 212009127). Nucleotides 945773 to 946654 (open reading frame TON_{—}1022) encodes a respiratory-chain NADH dehydrogenase, subunit 1 (Genbank accession number ACJ16510.1, GI number 212009128). Nucleotides 946658 to 948280 (open reading frame TON_{—}1023) encodes a hydrogenase 4, subunit 5 (Genbank accession number ACJ16511.1, GI number 212009129). Nucleotides 948282 to 948778 (open reading frame TON_{—}1023-1) encodes a membrane bound hydrogenase subunit N (this sequence was not annotated as a gene in the original genome sequence; it is encoded by nucleotides 948282 to 948778 in Genbank accession number CP000855, GI number 212008101). Nucleotides 948784 to 949359 (open reading frame TON_{—}1024) encodes a NADH dehydrogenase (ubiquinone) (Genbank accession number ACJ16512.1, GI number 212009130). Nucleotides 949349 to 949633 (open reading frame TON_{—}1025) encodes a hypothetical Na+/H+ antiporter MnhF subunit (Genbank accession number ACJ16513.1, GI number 212009131). Nucleotides 949630 to 949989 (open reading frame TON_{—}1026) encodes a Na+/H+ antiporter subunit (Genbank accession number ACJ16514.1, GI number 212009132). Nucleotides 949982 to 950236 (open reading frame TON_{—}1027) encodes a protein available at Genbank accession number ACJ16515.1, GI number 212009133. Nucleotides 950237 to 951007 (open reading frame TON_{—}1028) encodes a hypothetical multisubunit Na+/H+ antiporter, MnhB subunit (Genbank accession number ACJ16516.1, GI number 212009134). Nucleotides 951000 to 951404 (open reading frame TON_{—}1029) encodes a Hypothetical integral membrane protein (Genbank accession number ACJ16517.1, GI number 212009135). Nucleotides 951401 to 951886 (open reading frame TON_{—}1030) encodes a Hypothetical integral membrane protein (Genbank accession number ACJ16518.1, GI number 212009136). Nucleotides 951897 to 953459 (open reading frame TON_{—}1031) encode a Hydrogenase 4, subunit 3 (Genbank accession number ACJ16519.1, GI number 212009137). In one embodiment, the polypeptides that make up a complex having CODH activity is, or is structurally similar to, a reference polypeptide that includes the amino acid sequence of the polypeptides disclosed at these Genbank accession numbers.

The skilled person will recognize that the polypeptides that make up a complex having CODH activity can be compared to polypeptides from other extreme thermophiles, such as hyperthermophiles, using readily available algorithms such as Clustl to identify conserved regions of the polypeptides. Using this information the skilled person will be able to readily predict with a reasonable expectation that certain conservative substitutions to the amino acid sequence of the polypeptides disclosed at these Genbank accession numbers will not decrease activity of the polypeptide.

The metabolic pathway described herein is introduced into a microbial cell using genetic engineering techniques. While certain embodiments are described using P. furiosus, the microbes and methods of use are not limited to P. furiosus and there are a number of other options for microbes suitable for engineering to produce alcohols from organic acids and for use in the methods described herein. Examples of microbes that can be genetically engineered to include the metabolic pathway described herein include, but are not limited to, Pyrococcus furiosus, members of the genus Thermococcus including T. onnurineus, members of the genus Caldicellulosiruptor, including C. bescii, members of the genus Clostridium, including C. thermocellum, members of the genera Thermoanaerobacter, Thermoanaerobacterium and Moorella, including M. thermoacetica. If necessary, a coding region encoding an enzyme described herein can be modified using routine methods to reflect the codon usage bias of a microbial host cell to optimize expression of a polypeptide.

A genetically engineered cell contains one or more exogenous polynucleotides that have been created through standard molecular cloning techniques to bring together genetic material that is not natively found together. For example, a microbe is a genetically engineered microbe by virtue of introduction of an exogenous polynucleotide. DNA sequences used in the construction of recombinant DNA molecules can originate from any species. For example, bacterial DNA may be joined with fungal DNA. Alternatively, DNA sequences that do not occur anywhere in nature may be created by the chemical synthesis of DNA, and incorporated into recombinant molecules. Proteins that result from the expression of recombinant DNA are often termed recombinant proteins. Examples of recombination are described in more detail below and may include inserting foreign polynucleotides into a cell, inserting synthetic polynucleotides into a cell, or relocating or rearranging polynucleotides within a cell. General laboratory methods for introducing and expressing or overexpressing native and nonnative proteins such as enzymes in many different cell types (including bacteria and archaea) are routine and known in the art; see, e.g., Sambrook et al, Molecular Cloning: A Laboratory Manual, Cold Spring Harbor Laboratory Press (1989), and Methods for General and Molecular Bacteriology, (eds. Gerhardt et al.) American Society for Microbiology, chapters 13-14 and 16-18 (1994).

The introduction of the novel biosynthetic pathway for the production of an alcohol from an organic acid into a cell involves expression or overexpression of an AOR polypeptide and/or an AdhA polypeptide. An enzyme is “overexpressed” in a genetically engineered microbe when the enzyme is expressed at a level higher than the level at which it is expressed in a comparable wild-type cell. In cells that do not express a particular endogenous enzyme, or in cells in which the enzyme is exogenous (i.e., the enzyme is not native to the cell), any level of expression of that enzyme in the cell is deemed an “overexpression” of that enzyme for purposes of the present invention.

As will be appreciated by a person of skill in the art, overexpression of an enzyme can be achieved through a number of molecular biology techniques. For example, overexpression can be achieved by introducing into the host cell one or more copies of a polynucleotide encoding the desired enzyme. The polynucleotide encoding the desired enzyme may be endogenous or exogenous to the host cell. Typically, the polynucleotide is introduced into the cell using a vector. The polynucleotide may be circular or linear, single-stranded or double stranded, and can be DNA, RNA, or any modification or combination thereof. The vector can be any molecule that may be used as a vehicle to transfer genetic material into a cell. Examples of vectors include plasmids, viral vectors, cosmids, and artificial chromosomes, without limitation. Examples of molecular biology techniques used to transfer nucleotide sequences into a microorganism include, without limitation, transfection, electroporation, transduction, and transformation. These methods are routine and known in the art. Insertion of a vector into a target cell is usually called transformation for bacterial cells and transfection for eukaryotic cells, however insertion of a viral vector is often called transduction. The terms transformation, transfection, and transduction, for the purpose of the present invention, are used interchangeably herein.

In one embodiment, the vector is an expression vector. An “expression vector” or “expression construct” is any vector that is used to introduce a specific polynucleotide into a target cell such that once the expression vector is inside the cell, the protein that is encoded by the polynucleotide is produced by the cellular transcription and translation machinery. Typically, an expression vector includes regulatory sequences operably linked to the polynucleotide encoding the desired enzyme. Regulatory sequences are common to the person of the skill in the art and may include for example, an origin of replication, a promoter sequence, and/or an enhancer sequence.

An expression vector may optionally include a promoter that results in expression of an operably linked coding region during growth in anaerobic conditions. In one embodiment, a suitable promoter causes expression of an operably linked coding region at temperatures of at least 30° C., at least 40° C., at least 50° C., at least 60° C., at least 70° C., at least 80° C., at least 90° C., or up at 100° C. In one embodiment, a suitable promoter causes expression of an operably linked coding region at temperatures of no greater than 90° C., no greater than 80° C., no greater than 70° C., no greater than 60° C., no greater than 50° C., no greater than 40° C. or no greater than 30° C. In one embodiment, a suitable promoter causes expression of an operably linked coding region at temperatures between 30° C. and 100° C., between 50° C. and 100° C., between 60° C. and 90° C., or between 70° C. and 80° C.

The promoter useful in methods described herein may be, but is not limited to, a constitutive promoter, a temperature sensitive promoter, a non-regulated promoter, or an inducible promoter. A constitutive promoter drives expression of an operably linked coding region in a microbe when cultured at the temperatures described herein. The expression of a coding region operably linked to a constitutive promoter occurs at both high and low incubation temperatures, and the level of expression does not change substantially when expression at higher and lower incubation temperatures is compared. An example of a constitutive promoter is Pₛₗₚ, a P. furiosus promoter of the highly expressed S-layer protein (Chandrayan et al., 2012. J. Biol. Chem., 287:3257-3264). Other examples of constitutive promoters include P_{gdh}, Pₚₑₚ and P_{porγ}, which are promoters in both P. furiosus and T. kodakarensis of the highly expressed glutamate dehydrogenase, phosphoenolpyruvate synthase and pyruvate ferredoxin oxidoredutase subunit γ, respectively (for example, see Lipscomb et al. 2011. Appl. Environ. Microbiol. 77:2232-2238; Chandrayan et al., 2012. J. Biol. Chem., 287:3257-3264). Examples of useful promoters are also disclosed in Adams et al. (WO 2014/039879) and Kelly et al. (US Published Patent Application 2014/0248687).

The promoter may be a temperature sensitive promoter. In one embodiment, a temperature sensitive promoter drives expression of an operably linked coding region in a microbe at a greater level during incubation at low temperatures when compared to expression during incubation at high temperature. Such a promoter is referred to herein as a “cold shock” promoter. A cold shock promoter is induced at temperatures lower than the optimum growth temperature (Tₒₚₜ) of a microbe. In one embodiment, a cold shock promoter is induced when a microbe is cultured at a temperature of no greater than 75° C., no greater than 70° C., no greater than 65° C., no greater than 60° C., no greater than 55° C., no greater than 50° C., no greater than 45° C., no greater than 40° C., or no greater than 35° C. In one embodiment, a cold shock promoter is induced when a microbe is cultured at a temperature between 35° C. and 45° C., between 40° C. and 50° C., between 45° C. and 55° C., between 50° C. and 60° C., between 55° C. and 65° C., between 60° C. and 70° C., or between 65° C. and 75° C. Induction of a cold shock promoter in a genetically engineered microbe may result in an upregulation of expression of an operably linked coding region by at least 10-fold, at least 15-fold, at least 20-fold, at least 25-fold, or at least 30-fold compared to expression of the same operably linked coding region during growth of the genetically engineered microbe at its Tₒₚₜ.

Examples of cold shock promoters include those operably linked to the coding regions of P. furiosus described by Weinberg et al. (2005, J. Bacteriol., 187:336-348) and Kelly et al. (WO 2013/067326). A promoter is present in the region immediately upstream of the first codon of a coding region. In one embodiment, at least 150 nucleotides upstream to at least 200 nucleotides upstream of the first codon of the operably linked coding region includes the promoter. The size of the region that includes a promoter may be limited by the presence of an upstream coding region such as a start codon (for a coding region on the opposite strand) or a stop codon (for a coding region on the same strand). Identifying promoters in microbes, including hyperthermophilic archaea and extreme thermophilic archaea, is routine (see, for example, Lipscomb et al., 2009, Mol. Microbiol., 71:332-349). Other archaea contain homologues of the coding regions described by Weinberg et al. (2005, J. Bacteriol., 187:336-348), and the promoters of such homologues can be evaluated for induced expression at lower temperatures. Cold sock promoters may be produced using recombinant techniques.

In one embodiment, a temperature sensitive promoter drives expression of an operably linked coding region in a microbe at a decreased level during incubation at low temperatures when compared to expression during incubation at high temperature. Such a promoter is referred to herein as a “cold repressed” promoter. As described herein, a genetically engineered microbe may be used to produce a product; however, the microbe may normally produce an endogenous enzyme that uses the product or an intermediate leading to the product. The use of a cold repressed promoter is advantageous in such an embodiment. The genetically engineered microbe may be modified to decrease the production of the endogenous enzyme. For instance, a microbe may be genetically engineered by removing the promoter driving expression of an endogenous enzyme and replacing it with a cold repressed promoter.

A vector may include a ribosome binding site (RBS) and a start site (e.g., the codon ATG) to initiate translation of the transcribed message to produce the polypeptide (Adams et al., WO 2014/039879 and Kelly et al., US Published Patent Application 2014/0248687). Like other regulatory sequences, a RBS may be heterologous with respect to a host cell. When expressing an exogenous polynucleotide in P. furiosus, it was found that in some embodiments the RBS could be modified to help with expression. A consensus RBS that may be used in P. furiosus is TAGTGGAGGATA (SEQ ID NO:37), where the underlined portion of the consensus RBS is usually at nucleotide position −10 to −5 relative to the start codon of the operably linked coding region. Other examples of useful RBS sequences include, but are not limited to, GGTGATATGCAATG (SEQ ID NO:38), GGAGGTGGAGAAAATG (SEQ ID NO:39), GGAGGTTTGAAGATG (SEQ ID NO:40), GGAGGTGTGGGAAAATG (SEQ ID NO:41), and GGAGGGGGTGAGAGAGATG (SEQ ID NO:42), where the predicted RBS is underlined and the first codon of an operably linked coding region is a double underlined ATG.

A vector may also include a termination sequence to end translation. A termination sequence is typically a codon for which there exists no corresponding aminoacetyl-tRNA, thus ending polypeptide synthesis. The polynucleotide used to transform the host cell can optionally further include a transcription termination sequence. Transcription termination sequences are known, and one example is AATCTTTTTTAG (SEQ ID NO:43).

The polynucleotide encoding the desired enzyme can exist extrachromosomally or can be integrated into the host cell chromosomal DNA. More typically, extrachromosomal DNA is maintained within the vector on which it was introduced into the host cell. In many instances, it may be beneficial to select a high copy number vector in order to maximize the expression of the enzyme. Multiple vectors may be present in a single cell, and in such cases vectors with compatible origins of replication are used. Optionally, the vector may further contain a selectable marker. Certain selectable markers may be used to confirm that the vector is present within the target cell. Other selectable markers may be used to further confirm that the vector and/or transgene has integrated into the host cell chromosomal DNA. The use of selectable markers is common in the art and the skilled person will understand and appreciate the many uses of selectable markers.

A vector introduced into a host cell optionally includes one or more marker sequences, which typically encode a molecule that inactivates or otherwise detects or is detected by a compound in the growth medium. Certain selectable markers may be used to confirm that the vector is present within the target cell. Other selectable markers may be used to further confirm that the vector and/or transgene has integrated into the host cell chromosomal DNA. For example, the inclusion of a marker sequence may render the transformed cell resistant to an antibiotic, or it may confer compound-specific metabolism on the transformed cell. Examples of a marker sequence include, but are not limited to, sequences that confer resistance to kanamycin, ampicillin, chloramphenicol, tetracycline, streptomycin, and neomycin. Examples of nutritional markers useful with certain host cells, including extremophiles, are disclosed in Lipscomb et al. (US Published Patent Application 20120135411), and include, but are not limited to, a requirement for uracil, histidine, or agmatine.

Whether a cell expresses or overexpresses an enzyme of the pathway described herein can easily be determined by a skilled person using a basic in vitro or in vivo enzyme assay. Common methods for measuring the amount of the product of a reaction catalysed by an enzyme may include, without limitation, chromatographic techniques such as size exclusion chromatography, separation based on charge or hydrophobicity, ion exchange chromatography, affinity chromatography, or liquid chromatography. The genetically engineered cell will yield a greater activity than a wild-type cell in such an assay. Additionally, or alternatively, the amount of an enzyme can be quantified and compared by obtaining protein extracts from the genetically engineered cell and a comparable wild-type cell and subjecting the extracts to any of number of protein quantification techniques which are well known in the art. Methods of protein quantification may include, without limitation, spectrophotometric methods, SDS-PAGE, western blotting, and mass spectrometry.

A genetically engineered cell described herein expresses or overexpresses an AOR polypeptide. The host cell may or may not express an AOR endogenously. If it does not express an endogenous AOR, it is genetically engineered to express an AOR. In one embodiment, the AOR is overexpressed; i.e., the genetically engineered cell expresses an AOR at a level higher than the level of AOR in a comparable wild-type cell. Where a cell does not express an AOR endogenously, any expression of the AOR is considered to be “overexpression.” Determination of whether an AOR is expressed or overexpressed can easily be made by a skilled person using a basic in vitro or in vivo enzyme assays. A coding region encoding an AOR may be obtained from a suitable biological source, such as a bacterial cell or archaeal cell, using standard molecular cloning techniques.

A genetically engineered cell described herein expresses or overexpresses an AdhA polypeptide. The host cell may or may not express an AdhA endogenously. If it does not express an endogenous AdhA, it is genetically engineered to express an AdhA. In one embodiment, the AdhA is overexpressed; i.e., the genetically engineered cell expresses an AdhA at a level higher than the level of AdhA in a comparable wild-type cell. Where a cell does not express an AdhA endogenously, any expression of the AdhA is considered to be “overexpression.” Determination of whether an AdhA is expressed or overexpressed can easily be made by a skilled person using a basic in vitro or in vivo enzyme assays. A coding region encoding an AdhA may be obtained from a suitable biological source, such as a bacterial cell or archeal cell, using standard molecular cloning techniques.

Provided herein are methods for using a genetically engineered microbe. Prior to the genetically modified microbes described herein, ethanol fermentation pathways operated at moderate temperatures (30° C. to 60° C.), and no cellular method had been described for converting carboxylic acids to alcohols using sugars or carbon monoxide as the energy source. The use in some embodiments of a hyperthermophile as the genetically modified microbe provides useful advantages including reduced risk of contamination of cultures, as growth of most microbes is reduced, or non-existent. Another advantage is lowered costs for cooling and distillation of the alcohol(s) produced. The use of anaerobic conditions reduces the risks inherent in processing the alcohols that can be used as fuels, such as combustion. Moreover, many hyperthermophiles including Pyrococcus and Thermococcus have genomes of reduced complexity, and encode fewer polypeptides. The reduced complexity results in a more streamlined metabolism with fewer intermediates and decreased metabolic diversity. Hence, there is a decreased likelihood that there will be overlap between the metabolites and/or enzymes of the host with those in the engineered metabolic pathway.

The method includes culturing a genetically engineered microbe under conditions suitable for production of an alcohol. The genetically engineered microbe is cultured in the presence of one or more carboxylic acids. As described herein, the carboxylic acids may be aliphatic, branched-chain and/or aromatic, and serve as electron acceptors to produce the corresponding alcohol. Essentially any concentration of carboxylic acids may be used, provided the genetically modified microbe is tolerant to the level of the acid, and methods for determining the resistance of a microbe to any particular carboxylic acid or combination of carboxylic acids are known in the art and routine. It is expected that the amount of an alcohol produced, or a combination of alcohols produced, may in some embodiments be high enough that the genetically modified microbe is not tolerant and metabolism is reduced. Methods for determining the resistance of a microbe to any particular alcohol or combination of alcohols are known in the art and routine.

The conditions for culturing are preferably anaerobic. In one embodiment, the genetically engineered microbe is cultured at a temperature that allows the AOR and AdhA polypeptides to catalyze their respective reactions. Such temperatures may be at least 30° C., at least 40° C., at least 50° C., at least 60° C., at least 70° C., at least 80° C., at least 90° C., or up to 100° C., and no greater than 90° C., no greater than 80° C., no greater than 70° C., no greater than 60° C., no greater than 50° C., no greater than 40° C., or no greater than 30° C. In one embodiment, a culture temperature is between 30° C. and 90° C., between 50° C. and 80° C., or between 60° C. and 70° C.

The microbial cell may be an archaeal cell, for instance, a member of the genera Pyrococcus, for instance P. furiosus, P. abyssi, or P. horikoshii, or a member of the genera Thermococcus, for instance, T. kodakaraensis or T. onnurineus. The microbial cell may be a bacterial cell, such as a member of the genus Caldicellulosiruptor spp., for instance C. bescii, a member of the genus Thermoanaerobacter, a member of the genus Thermoanaerobacterium, for instance, a member of the genus Moorella, for instance M. thermoacetica, or a member of the genus Clostridium, for instance C. thermocelum.

The methods can be performed using any convenient manner. For instance, methods for growing microbial cells to high densities are routine and known in the art, and include batch and continuous fermentation processes.

A method for using a genetically engineered microbe may include addition of an energy source, a reductant, a carbon source, or a combination thereof. Carbon sources include carbohydrates, such as carbohydrates that can be extracted from cell walls, including hexoses (e.g., glucose), pentoses (e.g., xylose), and other sugars (e.g., cellobiose, maltose). In one embodiment, the method includes the use of a plant biomass material such as lignocellulose, including carbohydrates resulting from the breakdown of the crosslinking between the polysaccharides and lignin present in lignocellulose. Lignocellulose may include pectins, hemicelluloses, and/or cellulose.

In one embodiment, the method includes the addition of synthesis gas (also known as synthetic gas and syngas). Synthesis gas is a mixture of hydrogen, carbon dioxide, and carbon monoxide, and is considered a third-generation feedstock for biological production of alcohols. Such an embodiment is useful when the genetically modified microbe includes carbon monoxide dehydrogenase (CODH) activity. CODH activity oxidizes carbon monoxide and reduces ferredoxin, which is used at by an AOR in the conversion of an organic acid to its corresponding aldehyde (see FIG. 1). The use of CODH activity in a genetically modified microbe results in alcohol production that is limited only by factors such as acid or alcohol tolerance, and not by electron availability (Muller, 2014, PNAS USA, 111(49):17352-17353).

A method for using a genetically engineered microbe may also include recovery of the product produced by the genetically engineered microbe. The method used for recovery depends upon the product, and methods for recovering products resulting from microbial pathways, including fermentation, are known to the skilled person and used routinely. For instance, when the product is ethanol, the ethanol may be distilled using conventional methods. For example, after fermentation the product, e.g., ethanol, may be separated from the fermented slurry. The slurry may be distilled to extract the ethanol, or the ethanol may be extracted from the fermented slurry by micro or membrane filtration techniques.

The present invention is illustrated by the following examples. It is to be understood that the particular examples, materials, amounts, and procedures are to be interpreted broadly in accordance with the scope and spirit of the invention as set forth herein.

### Example 1

The archaeon Pyrococcus furiosus grows optimally near 100° C. (Fiala et al., 1986, Arch Microbiol, 145(1):56-61) by fermenting simple and complex sugars to acetate, carbon dioxide, and hydrogen gas (Sapra et al., 2003, Proc Natl Acad Sci USA, 100(13):7545-7550). P. furiosus has an unusual Emden-Meyerhof pathway for the conversion of glucose to pyruvate because reductant is channeled not to NADH but to the redox protein ferredoxin (Fd; FIG. 1) by glyceraldehyde-3-phosphate (GAP) Fd oxidoreductase (GAPOR). Reduced Fd is reoxidized by a membrane-bound, energy-conserving H₂-evolving hydrogenase (Sapra et al., 2003, Proc Natl Acad Sci USA, 100(13):7545-7550). Pyruvate produced by glycolysis is subsequently oxidized to acetyl-CoA by pyruvate Fd oxidoreductase (POR), and acetyl-CoA is converted by ATP-forming acetyl-CoA synthetase (ACS) to acetate. P. furiosus was recently metabolically engineered to generate end products other than acetate in a temperature-controlled manner without the need for chemical inducers. Lactate was produced from glucose, and 3-hydroxypropionate was produced from carbon dioxide and glucose, using heterologously expressed enzymes encoded by foreign genes obtained from microbes that grow near 75° C. (Basen et al., 2012, Mbio 3(2):e00053-e12); Keller et al., 2013, Proc Natl Acad Sci USA, 110(15):5840-5845). At 98° C., the foreign enzymes were inactive and the engineered P. furiosus strains generated acetate, but near 70° C., the engineered strains produced either lactate or 3-hydroxypropionate instead.

The microbial production of ethanol (bioethanol) is a massive commercialized technology. Though alcohols with longer carbon chains are chemically much better suited for current transportation needs, their biotechnological production remains challenging. Here we have engineered the model hyperthermophile Pyrococcus furiosus to produce various alcohols from their corresponding organic acids by constructing a synthetic route termed the AOR/AdhA pathway (Basen et al., 2014, PNAS USA, 111(49):17618-17623). This synthetic pathway is not known in nature and is fundamentally different from those previously known. To the inventors' knowledge, this study is also the first example of significant alcohol formation in an archaeon, emphasizing the biotechnological potential of novel microorganisms. Moreover, the inventors show that carbon monoxide and hydrogen (syngas) can be used as the driving forces for alcohol production. The application of the AOR/AdhA pathway in syngas-fermenting microorganisms is potentially a game-changing platform technology for the production of longer bioalcohols, especially in combination with carbon chain elongation pathways.

The primary goal here was to engineer P. furiosus to produce ethanol near 70° C., applying a similar approach. We found that, unexpectedly, the insertion of a primary alcohol dehydrogenase, AdhA, led to the production of not only ethanol but also of a variety of bioalcohols from their corresponding organic acids. We used gene deletion analysis and ¹³C labeling to elucidate the biochemical pathway, and hypothesize it might be a remnant of an ancient energy-conservation mechanism. Furthermore, a P. furiosus strain A/Codh was developed that expresses a multisubunit carbon monoxide dehydrogenase. That strain used carbon monoxide as the electron donor for organic acid reduction to bioalcohols, emphasizing the biotechnological versatility and potential of the new synthetic pathway.

### Results and Discussion

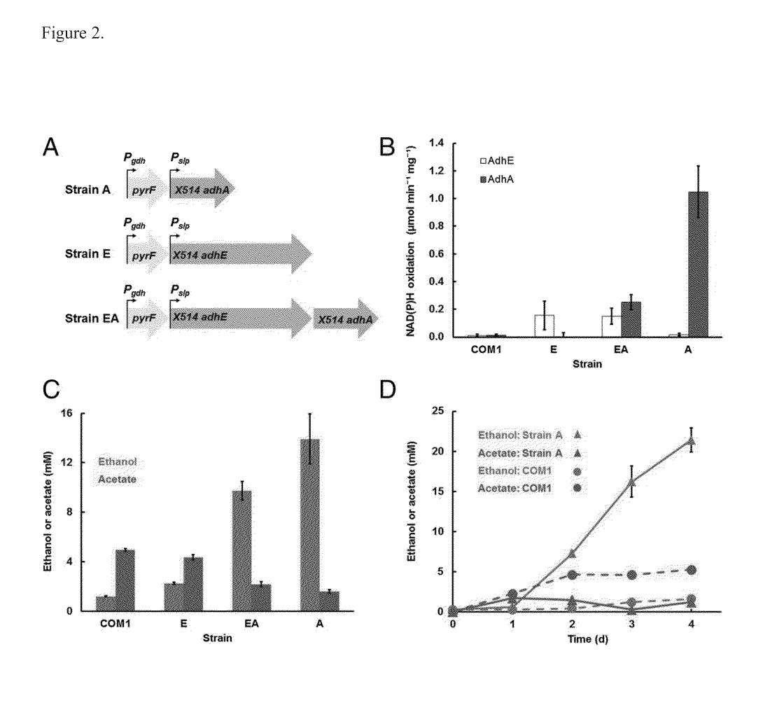

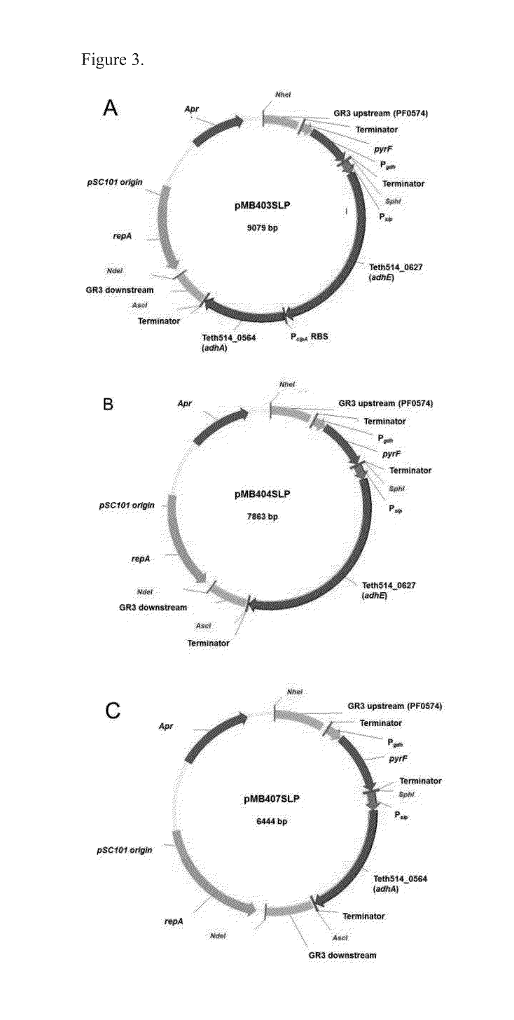

For ethanol production, the foreign genes to be inserted into P. furiosus encoded the bifunctional AdhE and the monofunctional AdhA enzymes, which generate ethanol from acetyl-CoA and acetaldehyde, respectively (Yao et al., 2010, J Mol Microbiol Biotechnol, 19(3):123-133; Pei et al., 2010, Metab Eng, 12(5):420-428). The genes were obtained from the thermophilic bacterium Thermoanaerobacter strain X514, which grows near 70° C. (Roh et al., 2002, Appl Environ Microbiol, 68(12):6013-6020). These genes were inserted, individually and in combination, into the P. furiosus genome (Lipscomb et al., 2011, Appl Environ Microbiol, 77(7):2232-2238), yielding strain E (containing adhE), strain A (containing adhA), and strain EA (containing adhE and adhA; FIGS. 2A and 3, and Table 2. As expected, when grown at 98° C., no AdhA or AdhE activity could be detected in cell extracts of any strain, although both activities were measured when the strains were grown at 72° C. (FIG. 2B). The activity of AdhA was lower in cell extracts of strain EA compared with those of strain A, possibly due to a lower expression level of adhA, because it is the second gene in the synthetic operon inserted into strain EA.

TABLE 2
Strains used.

| Strain | Strain | | | |
|---|---|---|---|---|
| Name | ID | Relevant genotype | Parent | Source |
| DSM 3638 | MW001 | Wild Type | n/a | (2) |
| COM1 | MW002 | ΔpyrF | DSM 3638 | (1) |
| COM1c | MW004 | ΔpyrF PgdhpyrF | COM1 | (3) |
| EA | MW606 | ΔpyrF PgdhpyrF | COM1 | This |
| | | Pslp(Teth514_0627; | | study |
| | | Teth5l4_0564) | | |
| E | MW607 | ΔpyrF PgdhpyrF | COM1 | This |
| | | PslpTeth514_0627 | | study |
| A | MW608 | ΔpyrF PgdhpyrF | COM1 | This |
| | | PslpTeth5l4_0564 | | study |
| AΔpyrF | MW610 | ΔpyrF | MW608 | This |
| | | PslpTeth514_0564 | | study |
| AΔaor | MW611 | Δaor ΔpyrF PgdhpyrF | MW610 | This |
| | | PslpTeth514_0564 | | study |
| A/Codh | MW258 | ΔpyrF PgdhpyrF | MW610 | This |
| | | PslpTeth5l4_0564; | | study |
| | | Pmbh1 | | |
| | | TON_1017-1031 | | |

(1) Lipscomb et al., 2011, Appl Environ Microbiol 77: 2232-2238.
(2) Fiala and Stetter, 1986, Arch Microbiol 145: 56-61.
(3) Thorgersen et al., 2014, Metab Eng 22: 83-88.

Surprisingly, however, at 72° C., strain E produced very little ethanol, only slightly more than the trace amounts produced by the parent strain (FIG. 2C). This result might be explained by high activity of the P. furiosus enzyme ATP-forming ACS, which competes with AdhE for the substrate acetyl-CoA (Thorgersen et al., 2014, Metab Eng, 22:83-88). Even more unexpected was that strain A generated very high amounts of ethanol (>20 mM), even more than that produced by strain EA (FIG. 2C) with very little acetate (<2 mM; FIG. 2D).

Because AdhA can generate ethanol only from acetaldehyde, and P. furiosus strain A does not contain bifunctional AdhE activity (FIG. 2B), acetyl-CoA is not likely to be the source of this acetaldehyde for ethanol production. Acetaldehyde could arise in P. furiosus from the decarboxylation of pyruvate, which was previously shown to be a significant side reaction of POR (Ma et al., 1997, Proc Natl Acad Sci USA, 94(18):9608-9613). Alternatively, it could arise by the reduction of acetate by P. furiosus aldehyde Fd oxidoreductase (AOR). This enzyme is highly expressed in P. furiosus when grown on sugars or peptides, and it has been shown to catalyze the reverse reaction in vitro, the oxidation of various aldehydes to their corresponding acid. It is thought that the in vivo function of AOR is to oxidize toxic aldehydes generated from the 2-ketoacids that are produced during sugar and peptide fermentation. However, this hypothesis has not been experimentally verified (Heider et al., 1995, J Bacteriol, 177(16):4757-4764).

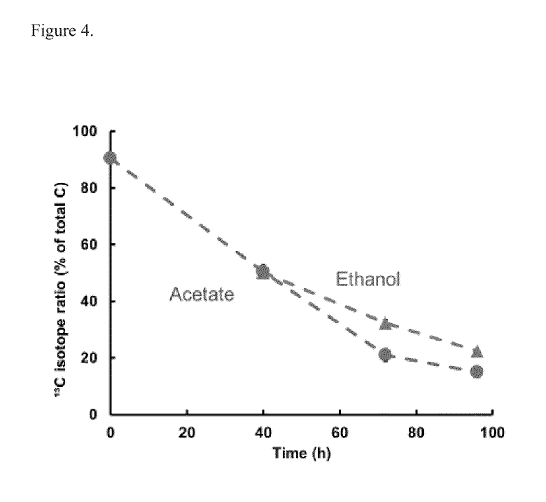

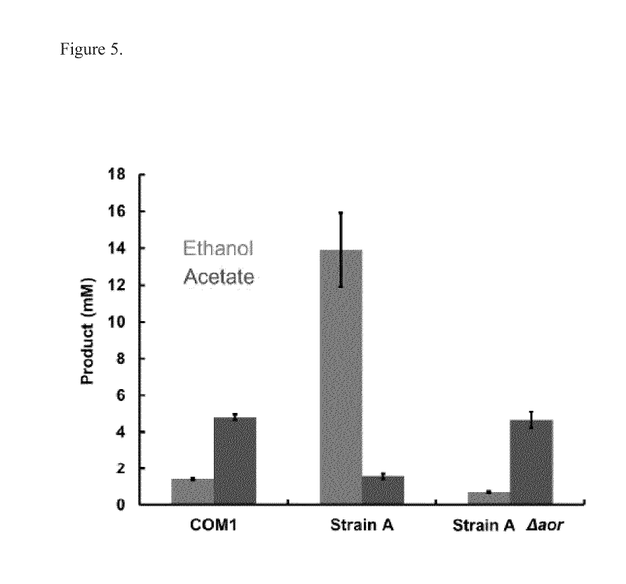

To distinguish between pyruvate or acetate as the source of acetaldehyde, ¹³C-labeled acetate was added to P. furiosus strain A growing at 72° C. on sugar (the disaccharide maltose), and the isotopic composition of the ethanol produced was analyzed. Approximately 50% of the ethanol formed after 40 h incubation contained the ¹³C label (FIG. 4); this can only occur if the acetaldehyde for ethanol production was derived from the added labeled acetate, which was subsequently diluted by unlabeled acetate produced from maltose degradation. To prove that AOR was responsible for reducing the acetate to acetaldehyde, the gene encoding AOR (PF0346) was deleted in strain A. As expected, the new P. furiosus strain A/Δaor, containing Thermoanaerobacter strain X514 AdhA but lacking the host's AOR, generated only trace amounts of ethanol from maltose, similar to that of the original parent strain (FIG. 5). It is not clear if AOR normally generates any acetaldehyde from acetate in wild-type P. furiosus (lacking AdhA) and, if so, how that the acetaldehyde is further metabolized. In any event, the properties of strain A call into question the previously proposed role of AOR (Heider et al., 1995, J Bacteriol, 177(16):4757-4764).

The proposed synthetic pathway for ethanol production in P. furiosus strain A is shown in FIG. 1. Acetate generated from glucose oxidation is reduced by AOR, and the acetaldehyde produced is reduced to ethanol by heterologously expressed AdhA. As indicated in FIG. 1, ethanol production from glucose is redox balanced. Reduced Fd for acetate reduction by AOR is supplied by POR and GAPOR (FIG. 1), whereas the NADPH for ethanol production by AdhA must also be generated from reduced Fd; this could occur either by ferredoxin NAD(P) oxidoreductase or from H₂ via the cytoplasmic hydrogenase (SHI) of P. furiosus. In addition, energy is conserved in the form of ATP by the ACS reaction. Consequently, this synthetic pathway theoretically converts 0.5 mol of glucose to 1 mol of ethanol and 1 mol of CO₂, according to Eq. 1:

0.5 glucose+ADP+Pi→ethanol+CO₂+ATP [1]

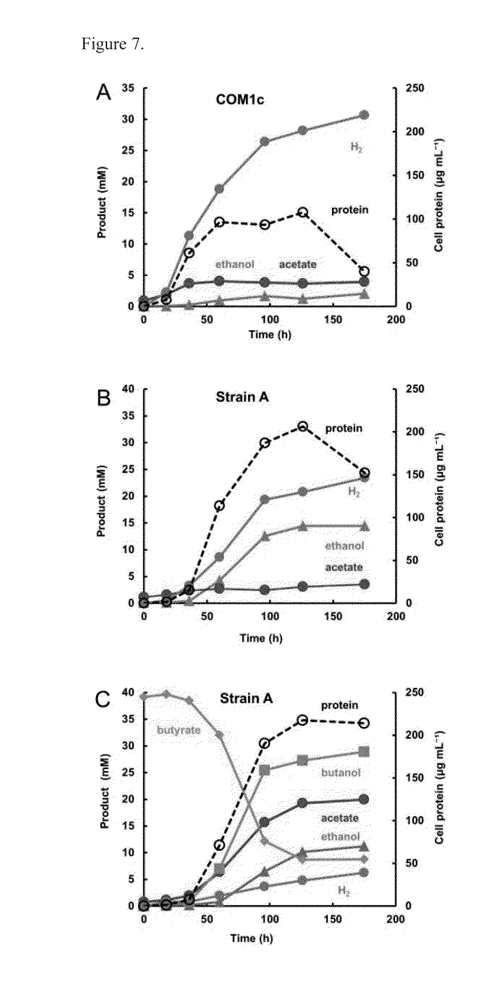

Because the synthetic pathway converted the added ¹³C-labeled acetate to ethanol, we investigated whether other exogenously supplied organic acids would similarly be converted to their corresponding alcohol by the AOR/AdhA pathway; this would seem likely because AOR has a very broad substrate specificity—it oxidizes the decarboxylated forms of keto acids derived from the transaminated derivatives of virtually all 20 amino acids (Roy et al., 2002, Met Ions Biol Syst, 39:673-697). Hence, when 40 mM butyrate was added to a culture of P. furiosus strain A at 72° C., almost 30 mM butanol was generated (FIGS. 26A and 7) with the reductant supplied by glucose, according to Eq. 2:

0.5 glucose+butyrate+ADP+Pi→acetate+butanol+CO₂+ATP. [2]

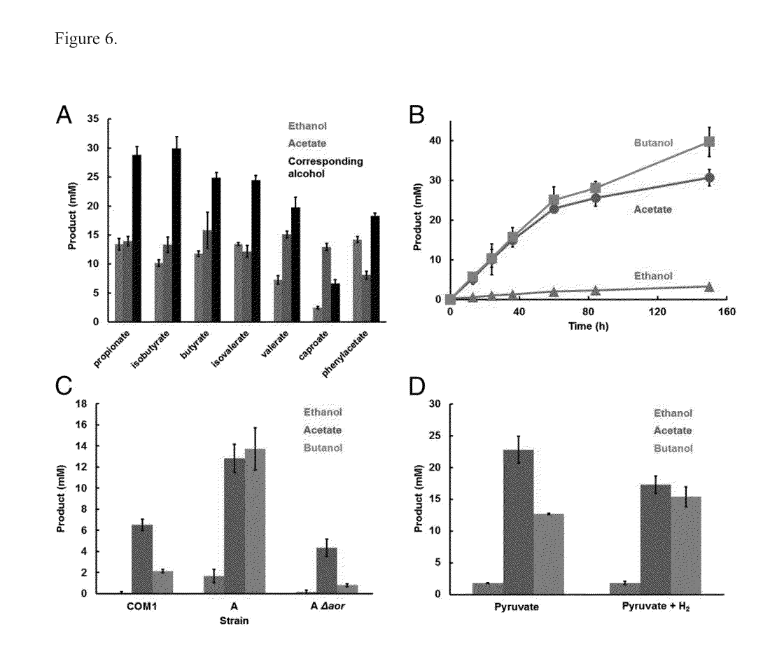

Similar results were obtained when propionate, isobutyrate, valerate, isovalerate, caproate, or phenylacetate were added to P. furious strain A, generating propanol, isobutanol, 1-pentanol, isoamylalcohol, 1-hexanol, and phenylethanol, respectively (FIG. 6A). When butyrate was added to strain A/Δaor, insignificant amounts of butanol were formed (FIG. 6C), once more demonstrating the essential role of AOR in alcohol formation. P. furiosus strain A must, therefore, metabolize the sugar (maltose) to provide reductant for the conversion of the added acid to the corresponding alcohol (FIG. 1 and Eq. 2). Consequently, one would expect acetate to be also generated as the oxidized end product, and this was the case (FIG. 6A). Furthermore, ethanol was produced by the reduction of the acetate generated from sugar oxidation according to Eq. 1 (FIG. 6A). As shown in FIG. 6B, with butyrate as the added acid, acetate and butanol were produced in a 1:1 ratio (Eq. 2). Because butyrate was provided in great excess (100 mM), only minimal amounts of ethanol were produced. Under growth conditions, almost 40 mM (3 g·L⁻¹) butanol was generated at a rate of 0.34 mmol·h⁻¹·g of protein.

P. furiosus can also grow with pyruvate as a carbon and energy source, and so pyruvate should be able replace maltose and supply reductant via POR for butyrate reduction, and this should also result in the formation of ATP (FIG. 1 and Eq. 3). As shown in FIG. 6D, this proved to be the case. The acetate:butanol ratio is predicted to be 2:1 for redox balance (Eq. 3), and this was confirmed experimentally (FIG. 3D). Hydrogen gas (H₂) could also be used as a source of reductant in addition to pyruvate (FIG. 1). Use of H₂ is predicted to result in the production of equimolar amounts of butanol and acetate (Eq. 4), and this was also demonstrated in vivo (FIG. 6D).

2 pyruvate+butyrate+2 ADP+2 Pi→2 acetate+butanol+2 CO₂+2 ATP [3]

pyruvate+butyrate+H₂+ADP+Pi→acetate+butanol+CO₂+ATP [4]

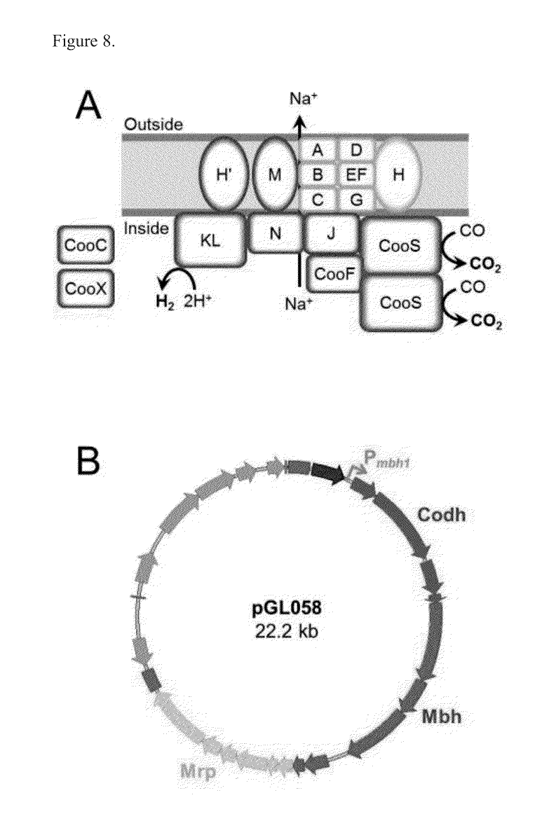

Hence, exogenous acid to alcohol conversion by P. furiosus strain A can be driven by the oxidation of glucose, pyruvate, or pyruvate plus H₂. Hydrogen gas cannot be used as the sole source of reductant for alcohol production, however, because its redox potential [H₂/H⁺, E₀′=−414 mV (pH 7.0)] is low enough to reduce NADP (E₀′=−320 mV) but not low enough to drive the reduction of P. furiosus Fd (E₀′=−480 mV) (Park et al., 1991, J Biol Chem, 266(29):19351-19356) for the AOR reaction. In contrast, carbon monoxide (CO) oxidation is a very low potential reaction (CO/CO₂, E₀′=−558 mV) that could potentially be coupled to Fd reduction for the AOR reaction, but P. furiosus does not metabolize CO (or any other C1 compound). Using a bacterial artificial chromosome, we genetically inserted into the chromosome of P. furiosus strain A the 16-gene operon encoding the complete carbon monoxide dehydrogenase/membrane-bound hydrogenase complex (CODH; FIG. 8) of the carboxydotrophic thermophile Thermococcus onnurineus, which oxidizes CO to H₂ and CO₂ at 80° C. (Schut et al., 2013, FEMS Microbiol Rev, 37(2):182-203; Yun et al., 2011, J Proteomics, 74(10):1926-1933). Remarkably, engineered P. furiosus strain A/Codh was able to use the strong reducing power of CO to produce high concentrations of the alcohol from the corresponding acid at 72° C. For example, the CO-dependent production of isobutanol (70 mM) from isobutyrate (105 mM) by strain A/Codh is shown in FIG. 9A.

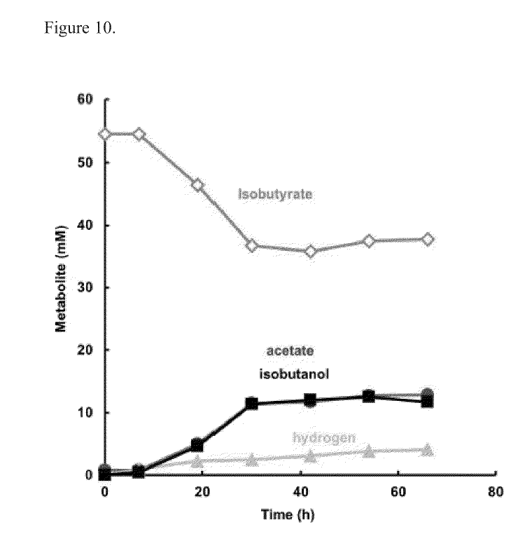

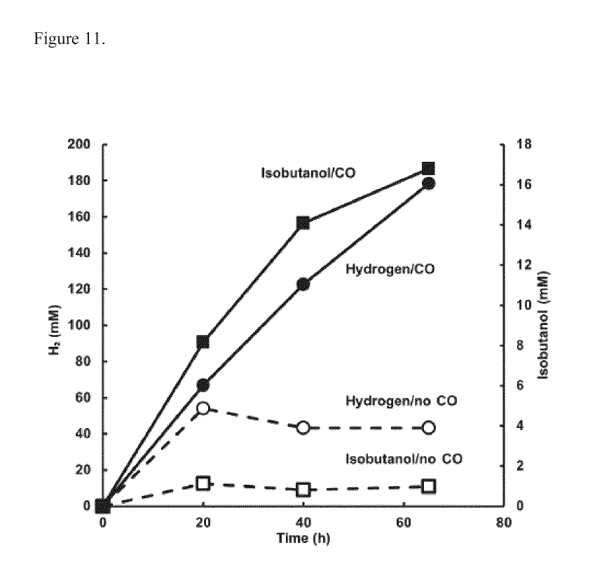

CO oxidation is not coupled to the production of acetate or any other organic compound (FIG. 9B), in contrast to the use of glucose or pyruvate to drive alcohol production by P. furiosus (FIG. 10). A cell suspension of strain A/Codh used CO as the only electron source to drive the AOR/AdhA pathway and convert isobutyrate to isobutanol, with no other products (except for CO₂ from CO oxidation; FIG. 11). Organic acids are therefore converted to the corresponding alcohol with minimal input of the host's energy metabolism (FIG. 9B). The CODH complex is thought to convert CO to H₂ and CO₂ without the involvement of intermediate electron carriers like ferredoxin in T. onnurineus, but this cannot be the case in P. furiosus. Because H₂ cannot be the sole source of reductant for organic acid production, T. onnurineus CODH expressed in P. furiosus must also reduce Fd directly, thereby allowed the resulting P. furiosus strain A/Codh to use CO as reductant for the reduction of organic acids by the AOR/AdhA pathway (FIG. 9B). CO-dependent conversion of acids to alcohols also results in H₂ production (FIG. 11); therefore, though CODH reduces P. furiosus Fd directly for the AOR reaction, the NADPH for the AdhA reaction is supplied at least in part via H₂ and SHI (FIG. 9B). However, no net H₂ production was observed until isobutanol production slowed down (FIG. 9A). The use of CO to provide the reducing equivalents to convert organic acids to their corresponding alcohols has great potential for using industrial syngas (CO and H₂) as both an energy source and a carbon source in microbial fermentations to convert organic acids generated from H₂ and CO₂ (Bengelsdorf et al., 2013, Environ Technol, 34(13-16):1639-1651) to the corresponding alcohol.

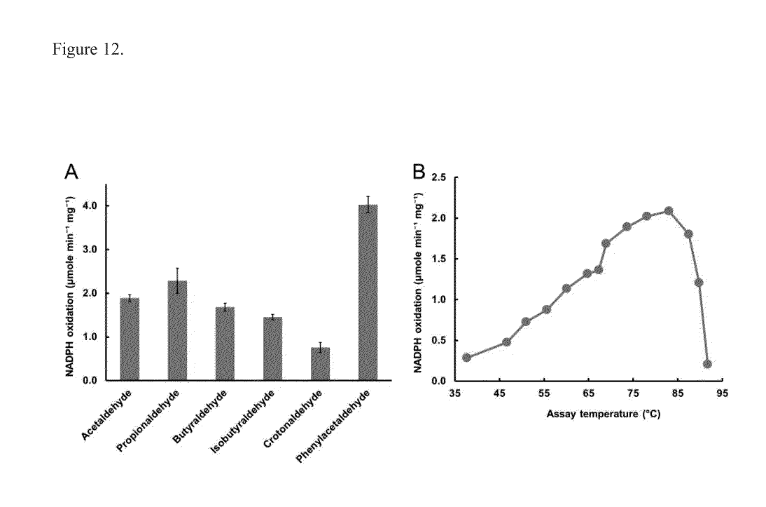

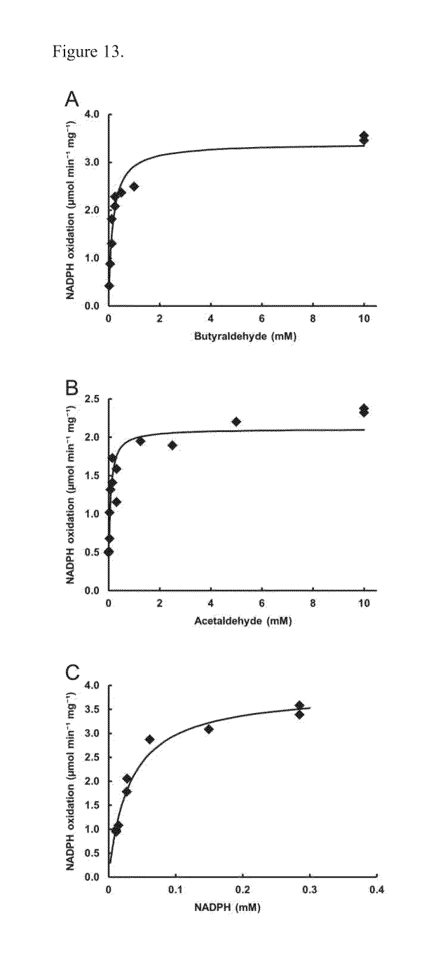

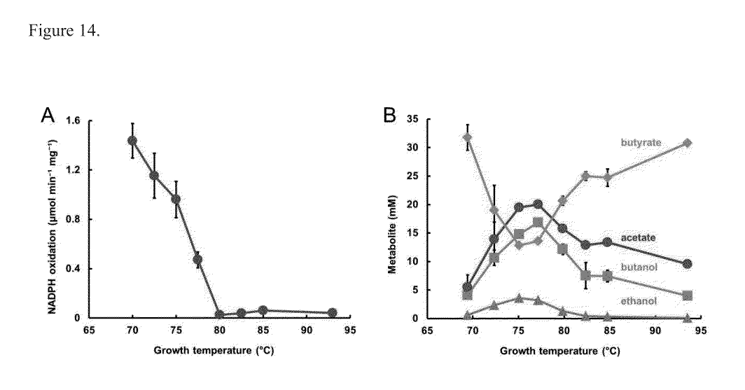

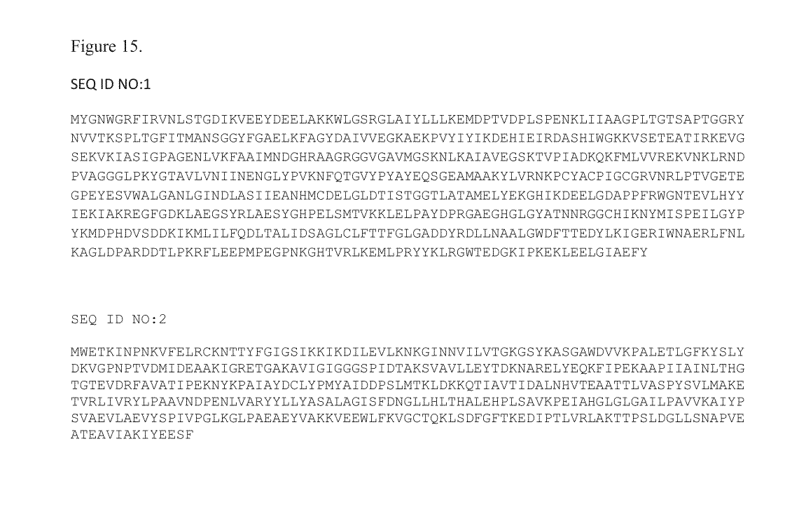

Hence, remarkably, with the introduction of a single foreign enzyme, encoded by adhA from Thermoanaerobacter strain X514, P. furiosus can convert glucose to ethanol as well as various organic acids to the corresponding alcohol. Moreover, with the introduction of second enzyme, CODH, CO can serve as the sole source of reductant for the reduction of structurally diverse acids, including aliphatic (C₂-C₆) and aromatic (phenyl acetate) derivatives. Accordingly, we found that recombinant AdhA (produced in P. furiosus) was able to reduce C₂-C₆ aldehydes and phenyl acetaldehyde to the corresponding alcohol, and thus it has a broad substrate spectrum, matching that of AOR (Roy et al., 2002, Met Ions Biol Syst, 39:673-697) (FIG. 12). AdhA also has high affinities for acetaldehyde, butyraldehyde, and NADPH, with apparent Michaelis constant (Kₘ) values of 63, 166, and 31 μM, respectively (FIG. 13), and so it is able to efficiently reduce the aldehydes generated by AOR. Maximal activity of AdhA was produced in P. furiosus when cells were grown in the 70 to 77° C. range, representing the optimal temperature for production and folding of the AdhA polypeptide (FIG. 14A); this correlates well with the optimum temperature for in vivo production of butanol from butyrate (70-80° C.; FIG. 14B). In fact, some butanol was still produced at 94° C., which corresponds to the upper limit for AdhA activity (FIG. 12).

Conversion of organic acids to the corresponding alcohols has been reported using the mesophilic anaerobic bacterium Clostridium ljungdahlii (Perez et al., 2013, Biotechnol Bioeng, 110(4):1066-1077) grown with CO as the energy source. Although the mechanism and pathway of carbon and electron flow has not been demonstrated, it likely proceeds via activation of organic acids to their CoA ester and CO-derived reducing equivalents are used to form alcohols from the acyl-CoA esters. In contrast, the synthetic AOR/AdhA pathway of P. furiosus for CO-dependent acid-to-alcohol conversion does not involve CoA derivatives (FIG. 9B). There are also reports suggesting that cell-free extracts and/or cell suspensions of the anaerobic bacteria Moorella thermoacetica and Clostridium formicaceticum catalyze a “through reduction” of acids to alcohols (Fraisse et al., 1988, Arch Microbiol, 150:381-386); Simon et al., 1987, Angew Chem Int Ed Engl, 26:785-787; White et al. 1989, Eur J Biochem, 184(1):89-96). These reactions were performed using CO, formate or H₂ as electron donors in the presence of artificial viologen dyes as electron carriers. However, the fermentation of glucose to ethanol via an AOR/AdhA-type pathway has not been shown previously. Moreover, direct involvement of AOR in microbial alcohol production from organic acids has not been previously demonstrated. It is the low potential fermentative pathway of P. furiosus, where ferredoxin is the sole electron acceptor of sugar oxidation that fuels the AOR reaction and alcohol production.

Conversion of organic acids to alcohols might have a primordial origin, because the synthesis of organic acids from CO₂ has been shown experimentally using metal catalysts (Huber et al., 1997, Science, 276(5310):245-247). Furthermore, C₂-C₆ carboxylic acids have been postulated to be the dominant carbon species in early earth hydrothermal vents based on thermodynamic considerations (Amend et al., 2013, Phil Trans R Soc B, 368:1622)). P. furiosus was isolated from a hot marine vent system (Fiala et al., 1986, Arch Microbiol, 145(1):56-61), and archaea in general are considered by some as the most primitive of all life forms. AOR might, therefore, be a remnant of an ancient pathway for energy conservation in a reducing early earth environment, where geochemically formed organic acids could have served as electron acceptors with carbon monoxide as the potential electron donor.

### Materials and Methods

Transformation of P. furiosus.

Escherichia coli XL1 Blue-MRF′ (Agilent Technologies) was used to amplify plasmid DNA. Plasmid DNA purification was performed using the StrataPrep Plasmid Miniprep Kit (Agilent). Extraction of DNA from P. furiosus, transformation of P. furiosus, screening of transformants, and strain purification were performed as previously described, except that the defined medium contained maltose (5 g·L⁻¹) instead of cellobiose as the sole growth substrate. The DNA sequence modification of isolated P. furiosus strains was verified by sequencing as previously described (Lipscomb et al., 2011, Appl Environ Microbiol, 77(7):2232-2238). Primers used to construct the strains, all plasmids, and all strains are listed in Tables 2 and 3.

TABLE 3
Primers used.

| Primer | Sequence (5′-3′) | Source |
|---|---|---|
| Pslp-SacII-F | gaatccccgcggaaatagatattatcggcaaacac | This study |
| | (SEQ ID NO: 11) | |
| Pslp-AdhE-R | cttgtaataaggtaggcatttttctccacctcccaataatc | This study |
| | (SEQ ID NO: 12) | |
| AdhE-Pslp-F | gattattgggaggtggagaaaaatgcctaccttattacaag | This study |
| | (SEQ ID NO: 13) | |
| AdhE-R2 | gtttcccacactgcatatcacctgccattattctccataggc | This study |
| | (SEQ ID NO: 14) | |
| AdhE-SphI-R | gcatgcggtaccagcctcctattattctccataggcttttcta | This study |
| | (SEQ ID NO: 15) | |
| AdhA-F2 | ctatggagaataatggcaggtgatatgcagtgtgggaaacaaaaataaatc | This study |
| | (SEQ ID NO: 16) | |
| AdhA-SphI-R | tacatgcatgcggtaccagcctcctattagaaagattcttcataaatc | This study |
| | (SEQ ID NO: 17) | |
| Pslp-SphI-F | tacatgcatgcaaatagatattatcggcaaacac | This study |
| | (SEQ ID NO: 18) | |
| AdhA-AscI-R | aggcgcgcctaaaaaagattttagaaagattcttcataaatcttg | This study |
| | (SEQ ID NO: 19) | |
| AdhE-AscI-R | aggcgcgcctaaaaaagattttattctccataggcttttc | This study |
| | (SEQ ID NO: 20) | |
| \dot{a}AdhA-Pslp-F | gattattgggaggtggagaaaagtgtgggaaacaaaaataaatcc | This study |
| | (SEQ ID NO: 21) | |
| SP2.055 | ttttctccacctcccaataatc | This study |
| | (SEQ ID NO: 22) | |
| AOR1 | gatagctagcgaaacttctctgcatcgtcaaga | This study |
| | (SEQ ID NO: 23) | |
| AOR2 | actcttcttttcaattaac | This study |
| | (SEQ ID NO: 24) | |
| AOR3 | agaggtcaccaacatatttattg | This study |
| | (SEQ ID NO: 25) | |
| AOR4 | tctacatatgatcgatctagaactttcagtattctcg | This study |
| | (SEQ ID NO: 26) | |
| AOR5 | ggaaataaaaagttaattgaaaagaagagtcccgggaagccgctaag | This study |
| | (SEQ ID NO: 27) | |
| AOR6 | caataaatatgttggtgacctctgcggccgcgtttaaacggc | This study |
| | (SEQ ID NO: 28) | |
| SP2.037 | gcctttcagcattgtatatgg | This study |
| | (SEQ ID NO: 29) | |
| SP2.088 | cttgaaaatgtttgaggaacacc | This study |
| | (SEQ ID NO: 30) | |
| SP.237 | ctgagggagatatggttaatatg | This study |
| | (SEQ ID NO: 31) | |
| SP.238 | ggaattactcacaaatgttccaacggccgcgtttaaacggc | This study |
| | (SEQ ID NO: 32) | |
| SP.239 | ttggaacatttgtgagtaattcc | This study |
| | (SEQ ID NO: 33) | |
| SP.243 | gaaccggaaaaagctggcatcgccaaacctccttaacatttg | This study |
| | (SEQ ID NO: 34) | |
| SP.244 | atgccagctttttccggttc | This study |
| | (SEQ ID NO: 35) | |
| SP.245 | catattaaccatatctccctcagacaacccattgatagtcatgtgc | This study |
| | (SEQ ID NO: 36) | |

Construction of adhA-Containing Strains A, E, EA, and A/Codh.

P. furiosus strain COM1 (Lipscomb et al., 2011, Appl Environ Microbiol, 77(7):2232-2238) served as the parent strain for genetic manipulations for the heterologous expression of the bifunctional aldehyde/alcohol dehydrogenase AdhE (Teth514 0627; GeneID: 5876124) and the primary alcohol dehydrogenase AdhA (Teth514 0564; GeneID 5877753) from Thermoanaerobacter strain X514 (Roh et al., 2002, Appl Environ Microbiol, 68(12):6013-6020). Genomic DNA was isolated according to Zhou et al. (Zhou et al., 1995, Int J Sys Bacteriol, 45(3):500-506). adhE was amplified from genomic DNA by PCR using the primer pairs AdhE-Pslp-F/AdhE-R2 (for construction of plasmid pMB303SLP) or AdhE-Pslp-F/AdhE-SphI-R (for construction of plasmid pMB304SLP). AdhA (for construction of plasmid pMB303SLP) was amplified using AdhA-F2/AdhA-SphI-R. The constitutive promoter Pₛₗₚ was amplified from genomic DNA of P. furiosus with the primer set Pslp-SacII-F/Pslp-adhE-R. Fusion products of Pₛₗₚ and adhE or adhE and adhA were obtained by overlap PCR. Products from overlap PCR were digested with the restriction enzymes SacII and SphI and ligated into plasmid vector pSPF300 as described previously (Basen et al., 2012, Mbio, 3(2):e00053-e12) to make plasmids pMB303SLP (containing adhE and adhA under control of Pslp) and pMB304SLP (containing only adhE under control of Pslp). pMB303SLP contained a ribosomal binding site of the cold-induced protein CipA (P_{CipA} RBS, 16 bases) between adhE and adhA. pMB303SLP and pMB304SLP were used for transformation of P. furiosus strain ΔpdaD.

For transformation of P. furiosus strain COM1, the Pₛₗₚ-adhE-adhA or Pₛₗₚ-adhE fusions were amplified from pMB303SLP and pMB304SLP using the primer pairs Pslp-SphI-F/AdhA-AscI-R or Pslp-SphI-F/AdhE-AscI-R, respectively, and additionally introducing the hpyA1 terminator T hpyA1 (Lipscomb et al., 2011, Appl Environ Microbiol, 77(7):2232-2238). The resulting PCR products were digested with AscI and SphI, and then ligated into plasmid pGL007 (Keller et al., 2013, Proc Natl Acad Sci USA, 110(15):5840-5845) to make plasmids pMB403SLP and pMB404SLP. Plasmid pMB407SLP for construction of strain A (FIG. 2A) is derived from plasmid pMB403SLP. Using the primers AdhA-Pslp-F/SP2.055, everything but the adhE gene was amplified from plasmid pMB403SLP, and the PCR product was assembled to yield plasmid pMB407SLP using a Gibson Assembly Master Mix (NEB). All plasmids were digested with the restriction enzyme NdeI, and the resulting linear DNA was used to transform strain COM1 to yield strains EA, E, and A (FIG. 2A).

A linear DNA construct was used for a knockout of the aor gene (PF0346) in strain A to make strain AΔaor. First, the marker P_{gdh}pyrF was removed from strain A by selection on 5-fluoroacetic acid (Lipscomb et al., 2011, Appl Environ Microbiol, 77(7):2232-2238) to yield strain MW610. Then, primer pairs AOR1/AOR2 and AOR3/AOR4 were used to amplify 500-bp regions upstream and downstream of PF0346. AOR5/SP2.037 and SP2.088/AOR6 were used to amplify the marker P_{gdh}pyrF from pGL007 (Keller et al., 2013, Proc Natl Acad Sci USA, 110(15):5840-5845). The PCR products were combined by overlap PCR, and the resulting DNA fragment containing the marker P_{gdh}pyrF flanked by 500-bp regions upstream and downstream of PF0346 was used to transform strain A. The deletion was verified by PCR and sequence analysis.

The pGL058 plasmid containing the T. onnurineus Codh was constructed via Gibson Assembly (NEB) of the following fragments: the 8.8-kb backbone BAC vector containing the pyrF genetic marker and flanking homologous recombination regions targeting the intergenic space between convergent genes PF1232 and PF1233, amplified from pGL054 (Lipscomb et al., 2014, J Biol Chem 289(5):2873-2879) with primers SP.238 and SP.237; the 200-bp mbh1 (PF1423) promoter region of the membrane-bound hydrogenase gene cluster, amplified from P. furiosus genomic DNA with primers SP.239 and SP.243; and the 13.3-kb Codh gene cluster (TON_{—}1017-TON_{—}1031), amplified from T. onnurineus genomic DNA using primers SP.244 and SP.245 (FIG. 8). The pGL058 plasmid was linearized using the unique PvuI restriction site on the BAC vector backbone before transformation of P. furiosus strain MW610.

### Cultivation of the Strains and Alcohol Production Experiments.

Thermoanaerobacter strain X514 was cultivated at 65° C. on complex medium used for cultivation of thermophilic heterotrophic anaerobes (modified DSMZ 516 medium) with 5 g·L⁻¹ cellobiose as electron donor (Yang et al., 2009, Appl Environ Microbiol, 75(14):4762-4769). P. furiosus (DSM 3638) was routinely grown at the indicated temperatures with 5 g·L⁻¹ maltose and 2 g·L⁻¹ yeast extract as described previously (Basen et al., 2012, Mbio, 3(2):e00053-e12). A total of 20 μM uracil (Sigma Chemical) was added as needed (Lipscomb et al., 2011, Appl Environ Microbiol, 77(7):2232-2238). In temperature-switch experiments, cells were grown at 95° C. until mid- to late-exponential growth phase (0.5-1×10⁸ cells), then cooled to 72° C. and kept at this temperature for another 20-48 h as described previously (Basen et al., 2012, Mbio, 3(2):e00053-e12; Keller et al., 2013, Proc Natl Acad Sci USA, 110(15):5840-5845). Growth was followed by cell counting and determination of cell protein concentration.

### Cell Suspension Experiments.

P. furiosus strain A and strain A/Codh were grown at 72° C. for 4 d to reach high cell density (>1×10⁸ cells per milliliter), pelleted by centrifugation (6,000×g) for 10 min, and then resuspended in media (1/10 of the original culture volume to achieve a 10× concentration). Maltose, pyruvate (as the electron donor), and organic acids (as electron acceptors) were added in excess (>40-100 mM). To test the effect of hydrogen or CO as the electron donor, the argon headspace was replaced by either gas (2 bar).

### ¹³C-Acetate Conversion Experiment.

The 10-mL cultures of P. furiosus strain A were supplied with 8-mM double ¹³C-labeled sodium acetate (sodium acetate-¹³C₂; Sigma-Aldrich) in addition to 5 g·L⁻¹ (unlabeled) maltose and incubated at 72° C. Samples were taken from the cultures over a 4-d time course to study the change in the carbon isotope signature of acetate and ethanol. Samples (100 μL) of spent media in 2-mL glass vials were acidified by addition of 10 μL 2 M H₂SO₄. Acidified samples were heated on a hot plate until boiling, and 1- to 4-μL samples were removed and analyzed by GC-MS to obtain ¹³C/¹²C ratios for acetic acid and ethanol. This ratio was taken to be equal to the ratio of the measured abundances for masses 62 and 60 for acetic acid and masses 45 and 47 for ethanol. Significant amounts of mixed compounds (containing both ¹³C and ¹²C) were not detected. A helium mobile phase was used at a head pressure of 12 psi on an Alltech Econo-Cap 30 m×0.25 mm EC-WAX column (0.25-μM film) using a Hewlett Packard HP5890A GC with a Hewlett Packard 5971A electron ionization MS. The temperature was held at 40° C. for a 3-min solvent delay and then increased to 220° C. at a rate of 15° C./min where it was held for an additional minute. For acetic acid measurements in samples with high amounts of ethanol, the method was modified to begin at 100° C. to avoid overloading the MS detector with ethanol while still obtaining sufficient signal for acetic acid. Mass spectra were collected at m/z of 40-200 at a scan rate of four scans per second.

### Preparation of Cell Extracts and Enzyme Assays.

P. furiosus cells were harvested by centrifugation for 10 min at 6,000×g. Cells were lysed under anoxic conditions by osmotic shock in 50 mM Tris.HCl (pH 8.0) and 2 mM sodium dithionite, and additionally by a short sonication treatment (30 s, maximum 36 W). The lysis buffer contained 0.5 μg/mL DNase I (Sigma) to decrease the viscosity of the protein extract. Particles were removed by centrifugation at 30,000×g for 10 min to yield the whole-cell extracts (S30). The supernatant of the whole-cell extract subjected to ultracentrifugation at 100,000×g for 1 h yielded the cytoplasmatic protein fraction (S100). The protein content was determined using a standard Bradford assay. Whole-cell extracts and S100 were kept anoxic at all times, and enzyme activity assays were performed under reducing conditions in anoxic 50 mM Mops (pH 7.5) plus 2 mM DTT at 70° C. Unless noted otherwise, aldehyde dehydrogenase (E.C. 1.2.1.3) was determined by oxidation of NADH (0.2 mM) with acetaldehyde (1 mM) as the substrate, and alcohol dehydrogenase was determined by the oxidation of NADPH (0.2 mM) with butyraldehyde (1 mM) as the substrate. Absorption of both NADH and NADPH was measured at 340 nm (ε=6.22 M⁻¹·cm⁻¹), and NAD(P)H oxidation activities are given in micromoles per minute per milligram. Aldehyde ferredoxin oxidoreductase was measured by the oxidation of butyraldehyde (1 mM) with benzyl viologen (1 mM) as electron acceptor as described previously (Mukund et al., 1991, J Biol Chem, 266(22):14208-14216). Vₘₐₓ and Kₘ values of AdhA were calculated using nonlinear regression (nls function) in R (R Development Core Team, 2013, Available at www.R-project.org/). Standard Gibbs free energies ΔG^{o}′ were calculated from the free energies of formation ΔG_{f}^{o}, which were taken from Thauer et al. (Thauer et al., 1977, Bacteriol Rev, 41(1):100-180).

### Chemical Analyses.

Alcohols and organic acids were measured using an Agilent 7890A GC equipped with a Carbowax/20 m column and an FID detector. Ethanol and organic acids were also determined using the Megazyme Ethanol Assay Kit (Megazyme) and using a Waters HPLC model 2690 equipped with an Aminex HPX-87H column (300×7.8 mm; Bio-Rad) and a photodiode array detector (model 996; Waters), respectively. Hydrogen and CO were determined on a GC-8A gas chromatograph (Shimadzu) equipped with a thermal conductivity detector and a molecular sieve column (model 5A 80/100; Alltech) with argon as the carrier gas.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A genetically engineered microbe comprising a metabolic pathway for the production of an alcohol from an organic acid under suitable conditions, wherein the conditions comprise a temperature of at least 50° C., at least 60° C., or at least 70° C., and wherein the metabolic pathway comprises an enzyme having aldehyde ferredoxin oxidoreductase (AOR) activity and an enzyme having alcohol dehydrogenase (AdhA) activity.

2. The genetically engineered microbe of claim 1 wherein the enzyme having AOR activity is endogenous and the enzyme having AdhA activity is exogenous.

3. The genetically engineered microbe of claim 2 wherein a coding region encoding the exogenous enzyme is integrated into the chromosome.

4. The genetically engineered microbe of claim 1 wherein the metabolic pathway comprises reduction of the organic acid coupled to oxidation of ferredoxin.

5. The genetically engineered microbe of claim 1 wherein the genetically engineered microbe is an archaeon or a bacterium.

6. The genetically engineered microbe of claim 10 wherein the genetically engineered microbe is Pyrococcus furiosus, Thermococcus spp. Caldicellulosiruptor spp., Thermoanaerobacter spp., Thermoanaerobacterium spp., Moorella spp. or Clostridium spp.

7. The genetically engineered microbe of claim 1 wherein the organic acid is acetate, butyrate, propionate, isobutyrate, valerate, isovalerate, caproate, phenylacetate, benzoic acid, Lactate, or 3-Hydroxypropionate.

8. The genetically engineered microbe of claim 1 wherein the genetically engineered microbe further comprises a carbon monoxide dehydrogenase (CODH) activity.

9. The genetically engineered microbe of claim 8 wherein a complex of polypeptides has the CODH activity.

10. The genetically engineered microbe of claim 9 wherein the complex comprises 16 polypeptides, the 16 polypeptides comprising amino acid sequences filed in Genbank under accession numbers 212009123, 212009124, 212009125, 212009126, 212009127, 212009128, 212009129, nucleotides 948282 to 948778 of GI number 212008101, 212009130, 212009131, 212009132, 212009133, 212009134, 212009135, 212009136, and 212009137

11. The genetically engineered microbe of claim 1 wherein the conditions comprise an anaerobic environment.

12. A method for producing an alcohol comprising culturing the microbe of claim 1 under conditions suitable to produce an alcohol.

13. The method of claim 12 further comprising isolating the alcohol.

14. The method claim 12 wherein the culturing comprises an incubation temperature of at least 70° C.

15. The method of claim 1 wherein the culturing comprises a carbon source selected from a hexose carbohydrate, a pentose carbohydrate, or a combination thereof.

16. The method of claim 15 wherein the hexose carbohydrate comprises a glucose.

17. The method of claim 1 wherein the culturing comprises a plant biomass.

18. The method of claim 12 wherein the alcohol produced is Ethanol, Butanol, Propanol, Isobutanol, 1-Pentanol, Isoamylalcohol, 1-Hexanol, Phenylethanol, Benzyl alcohol, 1,2-Propanediol, or 1,3-Propanediol.


SEQUENCE LISTING (42 sequences)

SEQ ID NO: 1 - protein, 605 aa, Artificial
  peptide sequence of an aldehyde ferredoxin oxidoreductase (AOR)
MYGNWGRFIRVNLSTGDIKVEEYDEELAKKWLGSRGLAIYLLLKEMDPTVDPLSPENKLIIAAGPLTGTSAPTGGRYNVVTKSPLTGFITMANSGGYFGAELKFAGYDAIVVEGKAEKPVYIYIKDEHIEIRDASHIWGKKVSETEATIRKEVGSEKVKIASIGPAGENLVKFAAIMNDGHRAAGRGGVGAVMGSKNLKAIAVEGSKTVPIADKQKFMLVVREKVNKLRNDPVAGGGLPKYGTAVLVNIINENGLYPVKNFQTGVYPYAYEQSGEAMAAKYLVRNKPCYACPIGCGRVNRLPTVGETEGPEYESVWALGANLGINDLASIIEANHMCDELGLDTISTGGTLATAMELYEKGHIKDEELGDAPPFRWGNTEVLHYYIEKIAKREGFGDKLAEGSYRLAESYGHPELSMTVKKLELPAYDPRGAEGHGLGYATNNRGGCHIKNYMISPEILGYPYKMDPHDVSDDKIKMLILFQDLTALIDSAGLCLFTTFGLGADDYRDLLNAALGWDFTTEDYLKIGERIWNAERLFNLKAGLDPARDDTLPKRFLEEPMPEGPNKGHTVRLKEMLPRYYKLRGWTEDGKIPKEKLEELGIAEFY

SEQ ID NO: 2 - protein, 399 aa, Artificial
  peptide sequence of an alcohol dehydrogenase (AdhA)
MWETKINPNKVFELRCKNTTYFGIGSIKKIKDILEVLKNKGINNVILVTGKGSYKASGAWDVVKPALETLGFKYSLYDKVGPNPTVDMIDEAAKIGRETGAKAVIGIGGGSPIDTAKSVAVLLEYTDKNARELYEQKFIPEKAAPIIAINLTHGTGTEVDRFAVATIPEKNYKPAIAYDCLYPMYAIDDPSLMTKLDKKQTIAVTIDALNHVTEAATTLVASPYSVLMAKETVRLIVRYLPAAVNDPENLVARYYLLYASALAGISFDNGLLHLTHALEHPLSAVKPEIAHGLGLGAILPAVVKAIYPSVAEVLAEVYSPIVPGLKGLPAEAEYVAKKVEEWLFKVGCTQKLSDFGFTKEDIPTLVRLAKTTPSLDGLLSNAPVEATEAVIAKIYEESF

SEQ ID NO: 3 - protein, 601 aa, Halanaerobium hydrogeniformans
MGKIYEYQVLRVNLSDGEISRETIAGENVKKFLGGRGLASKMLYDEIDPKVDPLSPENKLIYASGLLTGTAASTGGRYMVITKGPLTGTIASSNSGGFFGAEMRFSGNDIIVFEGKAEKPVYLSLKGDKAELKDASHLWGKGVYDTTDMLIEEFGDEKARVSCIGPGGEKLVKFASIMNEKDRAAGRTGVGAVMGSKNLKAIVVKADEKQVEYFKDEMMEVVKKQTKTLKEDGVTGEGLPALGTKVLDNIINSHGAYPRNNFQYGTFADVEEVSGEALVEKGYLQGNKGCYGCPIRCGRHTKLPNGQEGEGPEYETGWAFGADCGVNDLNAIVQANFLCNDLGLDTISAGATIAAAMELYEKGYIDKDELENGPELKFGSSESVIYYTRQMAYREGFGDQMAEGSYRLAEAKGNAELSMSVKKQELPAYDPRGIQGQGLNYATSNRGGCHVRGYMVSPEVLGVPEKLDPLELKGKPEWVKIFQDLTAVIDSLGMCLFTSFALGLEDYTEIANAGTEFDFTPEEMLENGERIWNLERQFNLEAGITPDQDKLPKRFDEPLPDGTFRGSAAKYREILPEYYEIRGWQEDGWISDEKLSEMGLK

SEQ ID NO: 4 - protein, 601 aa, Geobacter sulfurreducens
MGSGWHGRVLRVDLAARRCSIEELNGTWAREFIGGRGLGSRYLVEEVAPDVDPLSPRNKLILATGPLTGTYGAANGRYVVVTKSPLTGTIASSNSGGYFPAELRFAGFDLIIVEGKAEKPVYLKIYNQKAELADASHLWGKTTYETEDMIRREFHGDAKVAAIGPAGEKMVRFACIVNDKHRAAGRSGVGAVMGSKNLKAVAVRGTGGVTMADPAAYREAARESYRLLKGHPVTAEGLPALGTAVLVNVINQSGALPTRNFQSGTFEGAEDISGERLAQTWLRRNKGCFGCVIACGRVTRLADPRWTESGEGPEYETLWALGAACGVSDLAAITKANYLCNEYGMDTITAGGTVACAMELFERGLLTEGEAGMPLRFGDGDALVRMFQLMGTAEGFGAKLGLGSWRLAESYGAPELSMTVKKQEFPAYDPRGIQGMALEYATSNRGACHVRGYMVSPEILGVPEKLDPAVTEGKAGWTKAFQDFTAVVDSAGICLFTTFAIGAPQVSAFLNAATGLGYSLEQLLEAGDRIWNLERQFNLKAGIDPTQDTLPPRLLSEPLPDGPMQGSVARLAEMLPDYYRARGWDEQGIPTDEKLKSLGLA

SEQ ID NO: 5 - protein, 605 aa, Pyrococcus furiosus; identical to SEQ ID NO: 1

SEQ ID NO: 6 - protein, 607 aa, Clostridium ljungdahlii
MYGYKGKVLRINLSSKTYIVEELKIDKAKKFIGARGLGVKTLFDEVDPKVDPLSPDNKFIIAAGPLTGAPVPTSGRFMVVTKSPLTGTIAIANSGGKWGAEFKAAGYDMIIVEGKSDKEVYVNIVDDKVEFRDASHVWGKLTEETTKMLQQETDSRAKVLCIGPAGEKLSLMAAVMNDVDRTAGRGGVGAVMGSKNLKAIVVKGSGKVKLFDEQKVKEVALEKTNILRKDPVAGGGLPTYGTAVLVNIINENGVHPVKNFQKSYTDQADKISGETLTKDCLVRKNPCYRCPIACGRWVKLDDGTECGGPEYETLWSFGSDCDVYDINAVNTANMLCNEYGLDTITAGCTIAAAMELYQRGYIKDEEIAADGLSLNWGDAKSMVEWVKKMGLREGFGDKMADGSYRLCDSYGVPEYSMTVKKQELPAYDPRGIQGHGITYAVNNRGGCHIKGYMVSPEILGYPEKLDRLAVEGKAGYARVFHDLTAVIDSLGLCIFTTFGLGAQDYVDMYNAVVGGELHDVNSLMLAGDRIWTLEKIFNLKAGIDSSQDTLPKRLLEEQIPEGPSKGEVHKLDVLLPEYYSVRGWDKNGIPTEETLKKLGLDEYVGKL

SEQ ID NO: 7 - protein, 399 aa, Thermoanaerobacter sp.; identical to SEQ ID NO: 2

SEQ ID NO: 8 - protein, 399 aa, Clostridium botulinum
MWESKMAINEIREIRSKTTVYLGVGAIEKIYDIASNLKNMNIDKVLIVTGRGAYRKTGAWEYVEKALEKENITYILYNEVTPNPTVDQVDEAARMGNELGAKAVIGIGGGSPIDAAKSVAILLEYKDKTARDIYEFKFTPEKAAPVIAINLTHGTGTEGDRFAVVSIPEKEYKPAIAYDCIYPLYAIDDPQLMVKLPIHQTCYVSVDAINHVVEASTSKVANPYTILLAKETVRLVSRYLPQALQHPGDLTARYYLTYASLIAGICFDNGLLHFTHALEHPLSGVKPDLSHGLGLGILLPAVIKEIYPSVSEVLADILSPIVCGLEGNPGEAEIVAKGIEKWLYSIGITEKLTDLGFKENDVEKLTKLAFETPSLDLLLSMAPIDADEKVVSNIFRNSL

SEQ ID NO: 9 - protein, 400 aa, Desulfotomaculum ruminis
MWEANIPIHEVREIRVKTTVFLGVGAIKKIHDIAAELKQKRGIDKVVILTGKGSYKSTGAWDHVSAALEKNGIQHVLYNKVTPNPTVDQVDEAAKMGKELGAQGVIAIGGGSPIDAAKSVAILLAYPGKTARDVYEFKFTPDQAVPIVAINLTHGTGTEADRFAVVSIPEKEHKPAIAYDCIYPLYSIDDPALMTKLPENQTRYVSIDAVNHIVEAATSKVASPFSIMLAKETVRLISEYLPKALAQPEDLTARYYLLYASLIAGVAFDNGMLHFTHALEHPLSAVKPDLAHGLGLAVILPAVVKQIYPSSAAVLADVFSPIIPGLKGVPDETEKAAKGIEQWLASVGITSKLKDLGFTEGDVEKLTDLAFNTPSLDLLLSLAPVPADRERVAAIYHDSL

SEQ ID NO: 10 - DNA, 35 bp, Artificial
  synthetic oligonucleotide primer
gaatccccgcggaaatagatattatcggcaaacac

SEQ ID NO: 11 - DNA, 41 bp, Artificial
  synthetic oligonucleotide primer
cttgtaataaggtaggcatttttctccacctcccaataatc

SEQ ID NO: 12 - DNA, 41 bp, Artificial
  synthetic oligonucleotide primer
gattattgggaggtggagaaaaatgcctaccttattacaag

SEQ ID NO: 13 - DNA, 42 bp, Artificial
  synthetic oligonucleotide primer
gtttcccacactgcatatcacctgccattattctccataggc

SEQ ID NO: 14 - DNA, 43 bp, Artificial
  synthetic oligonucleotide primer
gcatgcggtaccagcctcctattattctccataggcttttcta

SEQ ID NO: 15 - DNA, 51 bp, Artificial
  synthetic oligonucleotide primer
ctatggagaataatggcaggtgatatgcagtgtgggaaacaaaaataaatc

SEQ ID NO: 16 - DNA, 48 bp, Artificial
  synthetic oligonucleotide primer
tacatgcatgcggtaccagcctcctattagaaagattcttcataaatc

SEQ ID NO: 17 - DNA, 34 bp, Artificial
  synthetic oligonucleotide primer
tacatgcatgcaaatagatattatcggcaaacac

SEQ ID NO: 18 - DNA, 45 bp, Artificial
  synthetic oligonucleotide primer
aggcgcgcctaaaaaagattttagaaagattcttcataaatcttg

SEQ ID NO: 19 - DNA, 40 bp, Artificial
  synthetic oligonucleotide primer
aggcgcgcctaaaaaagattttattctccataggcttttc

SEQ ID NO: 20 - DNA, 45 bp, Artificial
  synthetic oligonucleotide primer
gattattgggaggtggagaaaagtgtgggaaacaaaaataaatcc

SEQ ID NO: 21 - DNA, 22 bp, Artificial
  synthetic oligonucleotide primer
ttttctccacctcccaataatc

SEQ ID NO: 22 - DNA, 33 bp, Artificial
  synthetic oligonucleotide primer
gatagctagcgaaacttctctgcatcgtcaaga

SEQ ID NO: 23 - DNA, 19 bp, Artificial
  synthetic oligonucleotide primer
actcttcttttcaattaac

SEQ ID NO: 24 - DNA, 23 bp, Artificial
  synthetic oligonucleotide primer
agaggtcaccaacatatttattg

SEQ ID NO: 25 - DNA, 37 bp, Artificial
  synthetic oligonucleotide primer
tctacatatgatcgatctagaactttcagtattctcg

SEQ ID NO: 26 - DNA, 47 bp, Artificial
  synthetic oligonucleotide primer
ggaaataaaaagttaattgaaaagaagagtcccgggaagccgctaag

SEQ ID NO: 27 - DNA, 42 bp, Artificial
  synthetic oligonucleotide primer
caataaatatgttggtgacctctgcggccgcgtttaaacggc

SEQ ID NO: 28 - DNA, 21 bp, Artificial
  synthetic oligonucleotide primer
gcctttcagcattgtatatgg

SEQ ID NO: 29 - DNA, 23 bp, Artificial
  synthetic oligonucleotide primer
cttgaaaatgtttgaggaacacc

SEQ ID NO: 30 - DNA, 23 bp, Artificial
  synthetic oligonucleotide primer
ctgagggagatatggttaatatg

SEQ ID NO: 31 - DNA, 41 bp, Artificial
  synthetic oligonucleotide primer
ggaattactcacaaatgttccaacggccgcgtttaaacggc

SEQ ID NO: 32 - DNA, 23 bp, Artificial
  synthetic oligonucleotide primer
ttggaacatttgtgagtaattcc

SEQ ID NO: 33 - DNA, 42 bp, Artificial
  synthetic oligonucleotide primer
gaaccggaaaaagctggcatcgccaaacctccttaacatttg

SEQ ID NO: 34 - DNA, 20 bp, Artificial
  synthetic oligonucleotide primer
atgccagctttttccggttc

SEQ ID NO: 35 - DNA, 46 bp, Artificial
  synthetic oligonucleotide primer
catattaaccatatctccctcagacaacccattgatagtcatgtgc

SEQ ID NO: 36 - DNA, 12 bp, Artificial
  ribosome binding site (RBS)
tagtggaggata

SEQ ID NO: 37 - DNA, 14 bp, Artificial
  ribosome binding site (RBS) and a start site
ggtgatatgcaatg

SEQ ID NO: 38 - DNA, 16 bp, Artificial
  ribosome binding site (RBS) and a start site
ggaggtggagaaaatg

SEQ ID NO: 39 - DNA, 15 bp, Artificial
  ribosome binding site (RBS) and a start site
ggaggtttgaagatg

SEQ ID NO: 40 - DNA, 17 bp, Artificial
  ribosome binding site (RBS) and a start site
ggaggtgtgggaaaatg

SEQ ID NO: 41 - DNA, 19 bp, Artificial
  ribosome binding site (RBS) and a start site
ggagggggtgagagagatg

SEQ ID NO: 42 - DNA, 12 bp, Artificial
  transcription termination sequence
aatcttttttag